SEQUENCE LISTING (4 sequences)

SEQ ID NO: 1 - DNA, 1554 bp, synthetic construct
  source 1..1554: mol_type = other DNA
atggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 2 - protein, 517 aa, synthetic construct
MALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 1560 bp, synthetic construct
  source 1..1560: mol_type = other DNA
gccaccatggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 4 - protein, 519 aa, synthetic construct
ATMALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0171803 A1
(43) Pub. Date: May 29, 2025

(54) METHODS AND COMPOSITIONS FOR CAR T CELL THERAPY

(71) Applicants: Endocyte, Inc., West Lafayette, IN (US); Purdue Research Foundation, West Lafayette, IN (US)

(72) Inventors: Philip Stewart LOW, West Lafayette, IN (US); Haiyan Chu, West Lafayette, IN (US); Yong Gu Lee, West Lafayette, IN (US)

(21) Appl. No.: 18/914,891

(22) Filed: Oct. 14, 2024

Related U.S. Application Data
(62) Division of application No. 16/092,054, filed on Oct. 8, 2018, now Pat. No. 12,144,850.
(60) Provisional application No. 62/320,183, filed on Apr. 8, 2016.
(60) Provisional application No. 62/323,971, filed on Apr. 18, 2016.

(51) Int. Cl. C12N 15/86; A61K 9/00; A61K 35/12; A61K 35/17; A61K 39/00; A61K 39/395; A61K 40/11; A61K 40/31; A61K 40/42; A61K 47/54; A61K 47/55; A61K 48/00; A61P 35/00; C07K 14/705; C07K 14/725; C07K 16/44

(52) U.S. Cl. CPC C12N 15/86; A61K 9/0019; A61K 35/12; A61K 35/17; A61K 39/0011; A61K 39/39558; A61K 40/11; A61K 40/31; A61K 40/4202; A61K 47/551; A61K 47/555; A61K 48/005; A61P 35/00; C07K 14/7051; C07K 14/70517; C07K 16/44; A61K 2039/505; A61K 2039/5156; A61K 2239/31; A61K 2239/38; A61K 2239/49; C07K 2317/622; C07K 2317/73; C07K 2317/92; C07K 2319/02; C07K 2319/03; C07K 2319/33; C12N 2740/15043; C12N 2740/16043

(57) ABSTRACT

The present disclosure relates to methods of treating a patient with a cancer by administering to the patient a composition comprising CAR T cells and a small molecule linked to a targeting moiety by a linker. The disclosure also relates to compositions for use in such methods.

### CROSS-REFERENCE TO RELATED APPLICATIONS

This application is a division of U.S. application Ser. No. 16/092,054, filed Oct. 8, 2018, which is a U.S. national stage application under 35 U.S.C. § 371(b) of International Application No. PCT/US2017/026618 filed Apr. 7, 2017, which claims priority under 35 U.S.C. § 119(e) to U.S. Provisional Application Ser. No. 62/320,183, filed Apr. 8, 2016, and U.S. Provisional Application Ser. No. 62/323,971, filed Apr. 18, 2016, all of which are incorporated herein by reference in their entirety.

### INCORPORATION BY REFERENCE OF SEQUENCE LISTING

This application contains a Sequence Listing which has been submitted electronically in ST26 format and hereby incorporated by reference in its entirety. Said ST26 file, created on Jan. 7, 2025, is named 1165168US2WO1.xml and is 8,726 bytes in size.

### TECHNICAL FIELD

The present disclosure relates to methods of treating a patient with a cancer by administering to the patient a composition comprising CAR T cells and administering to the patient a small molecule linked to a targeting moiety by a linker. The disclosure also relates to compositions for use in such methods.

### BACKGROUND

Immunotherapy based on adoptive transfer of lymphocytes (e.g., T cells) into a patient is a valuable therapy in the treatment of cancer and other diseases. Many important advancements have been made in the development of immunotherapies based on adoptive transfer of lymphocytes. Among the many different types of immunotherapeutic agents, one of the most promising of the immunotherapeutic agents being developed is T cells expressing chimeric antigen receptors (CAR T cells). The chimeric antigen receptor (CAR) is a genetically engineered receptor that is designed to target a specific antigen, for example, a tumor antigen. This targeting can result in cytotoxicity against the tumor, for example, such that CAR T cells expressing CARs can target and kill tumors via the specific tumor antigens.

First generation CARs are composed of a recognition region, e.g., a single chain fragment variable (scFv) region derived from an antibody for recognition and binding to the antigen expressed by the tumor, and an activation signaling domain, e.g., the CD3ζ chain of T cells can serve as a T cell activation signal in CARs. Although CAR T cells have shown positive results in vitro, they have had limited success in eliminating disease (e.g., cancer) in clinical trials. One problem has been the inability to prolong activation and expand the CAR T cell population in vivo.

To address this problem, a co-stimulation domain (e.g. CD137, CD28 or CD134) has been included in second generation CARs to achieve prolonged activation of T cells in vivo. Addition of a co-stimulation domain enhances the in vivo proliferation and survival of T cells containing CARs, and initial clinical data have shown that such constructs are promising therapeutic agents in the treatment of diseases, such as cancer.

Although improvements have been made in CAR T cell therapies, several problems remain. First, ‘off-target’ toxicity may occur due to normal cells that express the antigen targeted by the CAR T cells (e.g., a tumor-associated antigen). Second, unregulated CAR T cell activation may be found where the rapid and uncontrolled elimination of diseased cells (e.g., cancer cells) by CAR T cells induces a constellation of metabolic disturbances, called tumor lysis syndrome, in the case where a tumor is being treated, or cytokine release syndrome (CRS), which can be fatal to patients. Tumor lysis syndrome and CRS can result due to administered CAR T cells that cannot be easily regulated, and are activated uncontrollably. Accordingly, although CAR T cells show great promise as a tool in the treatment of diseases, such as cancer, additional CAR T cell therapies are needed that provide reduced off-target toxicity, and more precise control of CAR T cell activation.

### SUMMARY OF THE INVENTION

The present inventors have discovered methods of reducing off-target toxicity, and more precisely controlling CAR T cell activation, providing important advancements in CAR T cell therapy. In the various embodiments described herein, a small molecule ligand linked to a targeting moiety by a linker is used as a bridge between the cancer and the CAR T cells directing the CAR T cells to the cancer for amelioration of the cancer. In one embodiment, the “small molecule ligand” can be, for example, a folate, DUPA, an NK-1R ligand, a CAIX ligand, a ligand of gamma glutamyl transpeptidase, or a CCK2R ligand, each of which is a small molecule ligand that binds specifically to cancer cells (i.e., the receptor for these ligands is overexpressed on cancers compared to normal tissues).

In one embodiment, the “small molecule ligand” is linked to a “targeting moiety” that binds to the CAR expressed by CAR T cells. In various embodiments, the “targeting moiety” can be selected, for example, from 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester (PFP), tetrafluorophenyl ester (TFP), a knottin, a centyrin, and a DARPin.

The “targeting moiety” binds to the recognition region of the genetically engineered CAR expressed by CAR T cells. Accordingly, the recognition region of the CAR (e.g., a single chain fragment variable region (scFv) of an antibody) is directed to the “targeted moiety.” Thus, the small molecule ligand linked to a targeting moiety by a linker acts as a ‘bridge’ between the cancer and the CAR T cells directing the CAR T cells to the cancer for amelioration of the cancer.

In one illustrative embodiment, the inventors have discovered that varying the dose of the small molecule ligand linked to a targeting moiety by a linker (i.e., the bridge), can result in the ability to control CRS in vivo. In another embodiment, the inventors have discovered that varying the linker in the small molecule ligand linked to a targeting moiety (the bridge) can control CRS in vivo upon CAR T cell activation. In yet another embodiment, combinations of these methods can be used for precise control of CAR T cell activation and cytokine release in vivo. In another embodiment, affinity of the small molecule ligand for its receptor on the cancer can be altered to control CAR T cell activation, or to achieve specificity for the cancer avoiding toxicity towards normal tissues.

In one embodiment, a method of treatment of a cancer is provided. The method comprises i) administering to a patient a first dose of a compound, or a pharmaceutically acceptable salt thereof, wherein the compound comprises a small molecule ligand linked to a targeting moiety by a linker, ii) administering to the patient a CAR T cell composition wherein the CAR T cell comprises a CAR directed to the targeting moiety, ii) administering to the patient a second dose of the compound, or the pharmaceutically acceptable salt thereof, wherein the second dose is different than the first dose, and treating the patient to ameliorate the cancer.

In another embodiment, a method of treatment of a cancer is provided. The method comprises i) administering to the patient a first conjugate, or a pharmaceutically acceptable salt thereof, ii) administering to the patient a CAR T cell composition wherein the CAR T cell comprises a CAR directed to the targeting moiety, iii) administering to the patient a second conjugate, or a pharmaceutically acceptable salt thereof, wherein the first and the second conjugate each comprise a small molecule ligand linked to a targeting moiety by a linker and wherein the first conjugate and the second conjugate are different, and iv) treating the patient to ameliorate the cancer.

In yet another embodiment, a method of treatment of a cancer is provided. The method comprises i) administering to a patient a first dose of a first conjugate, or a pharmaceutically acceptable salt thereof, ii) administering to the patient a CAR T cell composition wherein the CAR T cell comprises a CAR directed to the targeting moiety, ii) administering to the patient a second dose of a second conjugate, or a pharmaceutically acceptable salt thereof, wherein the first conjugate and the second conjugate each comprise a small molecule ligand linked to a targeting moiety, wherein the first conjugate and the second conjugate are different, and wherein the first dose and the second dose are different, and iv) treating the patient to ameliorate the cancer.

In yet another illustrative embodiment, a CAR T cell comprising a nucleic acid comprising SEQ ID NO:1 is provided. In another aspect, a CAR T cell comprising a polypeptide comprising SEQ ID NO:2 is provided. In another embodiment, an isolated nucleic acid comprising SEQ ID NO:1 and encoding a chimeric antigen receptor is provided. In still another embodiment, a chimeric antigen receptor polypeptide comprising SEQ ID NO:2 is provided. In another aspect, a vector comprising SEQ ID NO:1 is provided. In another illustrative embodiment, a vector is provided comprising SEQ ID NO:1 wherein the vector is a lentiviral vector.

Several embodiments are also described by the following enumerated clauses:

1. A method of treatment of a cancer, the method comprising
i) administering to a patient a first dose of a compound, or a pharmaceutically acceptable salt thereof, wherein the compound comprises a small molecule ligand linked to a targeting moiety by a linker;
ii) administering to the patient a CAR T cell composition wherein the CAR T cell comprises a CAR directed to the targeting moiety;
ii) administering to the patient a second dose of the compound, or the pharmaceutically acceptable salt thereof, wherein the second dose is different than the first dose; and
iv) treating the patient to ameliorate the cancer.

2. A method of treatment of a cancer, the method comprising
i) administering to the patient a first conjugate, or a pharmaceutically acceptable salt thereof;
ii) administering to the patient a CAR T cell composition wherein the CAR T cell comprises a CAR directed to the targeting moiety;
iii) administering to the patient a second conjugate, or a pharmaceutically acceptable salt thereof,
wherein the first and the second conjugate each comprise a small molecule ligand linked to a targeting moiety by a linker and wherein the first conjugate and the second conjugate are different; and
iv) treating the patient to ameliorate the cancer.

3. A method of treatment of a cancer, the method comprising
i) administering to a patient a first dose of a first conjugate, or a pharmaceutically acceptable salt thereof;
ii) administering to the patient a CAR T cell composition wherein the CAR T cell comprises a CAR directed to the targeting moiety;
ii) administering to the patient a second dose of a second conjugate, or a pharmaceutically acceptable salt thereof,
wherein the first conjugate and the second conjugate each comprise a small molecule ligand linked to a targeting moiety, wherein the first conjugate and the second conjugate are different, and wherein the first dose and the second dose are different; and
iv) treating the patient to ameliorate the cancer.

4. The method of clause 2 or 3 wherein the linker in the first conjugate, or the pharmaceutically acceptable salt thereof, and the linker in the second conjugate, or the pharmaceutically acceptable salt thereof, are different.

5. The method of clause 2 or 3 wherein the linker in the first conjugate, or the pharmaceutically acceptable salt thereof, and the linker in the second conjugate, or the pharmaceutically acceptable salt thereof, are the same.

6. The method of any one of clauses 2 to 5 wherein the ligand in the first conjugate, or the pharmaceutically acceptable salt thereof, and the ligand in the second conjugate, or the pharmaceutically acceptable salt thereof, are different.

7. The method of any one of clauses 2 to 5 wherein the ligand in the first conjugate, or the pharmaceutically acceptable salt thereof, and the ligand in the second conjugate, or the pharmaceutically acceptable salt thereof, are the same.

8. The method of any one of clauses 2 to 7 wherein the targeting moiety in the first conjugate, or the pharmaceutically acceptable salt thereof, and the targeting moiety in the second conjugate, or the pharmaceutically acceptable salt thereof, are different.

9. The method of any one of clauses 2 to 7 wherein the targeting moiety in the first conjugate, or the pharmaceutically acceptable salt thereof, and the targeting moiety in the second conjugate, or the pharmaceutically acceptable salt thereof, are the same.

10. The method of any one of clauses 1 to 9 wherein the ligand is selected from a folate, DUPA, an NK-1R ligand, a CAIX ligand, a ligand of gamma glutamyl transpeptidase, and a CCK2R ligand.

11. The method of clause 10 wherein the ligand is a folate.

12. The method of clause 10 wherein the ligand is an NK-1R ligand.

13. The method of clause 10 wherein the ligand is DUPA.

14. The method of clause 10 wherein the ligand is a CCK2R ligand.

15. The method of clause 10 wherein the ligand is a ligand of gamma glutamyl transpeptidase.

16. The method of any one of clauses 1 to 15 wherein the targeting moiety is selected from 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester (PFP), tetrafluorophenyl ester (TFP), a knottin, a centyrin, and a DARPin.

17. The method of clause 16 wherein the targeting moiety is FITC.

18. The method of clause 16 wherein the targeting moiety is DNP.

19. The method of clause 16 wherein the targeting moiety is TNP.

20. The method of any one of clauses 1 to 19 wherein the linker comprises polyethylene glycol (PEG), polyproline, a hydrophilic amino acid, a sugar, an unnatural peptidoglycan, a polyvinylpyrrolidone, and/or pluronic F-127. 21. The method of clause 20 wherein the linker comprises PEG.

22. The method of any one of clauses 1 to 21 wherein the compound, or the pharmaceutically acceptable salt thereof, the first conjugate, or the pharmaceutically acceptable salt thereof, or the second conjugate or the pharmaceutically acceptable salt thereof, has the formula

B-L-T,

wherein B represents the small molecule ligand, L represents the linker, and T represents the targeting moiety, and wherein L comprises a structure having the formula

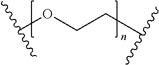

wherein n is an integer from 0 to 200.

23. The method of clause 22 wherein n is an integer from 0 to 150.

24. The method of clause 22 wherein n is an integer from 0 to 110.

25. The method of clause 22 wherein n is an integer from 0 to 20.

26. The method of clause 22 wherein n is an integer from 15 to 20.

27. The method of clause 22 wherein n is an integer from 15 to 110.

28. The method of any one of clauses 1 to 27 wherein the linker comprises PEG and the targeting moiety is FITC, or a pharmaceutically acceptable salt thereof.

29. The method of any one of clauses 1 to 28 wherein the dose of the compound, or the pharmaceutically acceptable salt thereof, the first conjugate, or the pharmaceutically acceptable salt thereof, or the second conjugate, or the pharmaceutically acceptable salt thereof, is about 10 nmol/kg to about 3000 nmol/kg of patient body weight.

30. The method of any one of clauses 1 to 29 wherein the dose of the compound, or the pharmaceutically acceptable salt thereof, the first conjugate, or the pharmaceutically acceptable salt thereof, or the second conjugate, or the pharmaceutically acceptable salt thereof, is about 50 nmol/kg to about 2000 nmol/kg of patient body weight.

31. The method of any one of clauses 1 to 30 wherein the dose of the compound, or the pharmaceutically acceptable salt thereof, the first conjugate, or the pharmaceutically acceptable salt thereof, or the second conjugate, or the pharmaceutically acceptable salt thereof, is about 100 nmol/kg to about 1000 nmol/kg of patient body weight.

32. The method of any one of clauses 1 to 31 wherein the dose of the compound, or the pharmaceutically acceptable salt thereof, the first conjugate, or the pharmaceutically acceptable salt thereof, or the second conjugate, or the pharmaceutically acceptable salt thereof, is about 100 nmol/kg to about 600 nmol/kg of patient body weight.

33. The method of any one of clauses 1 to 32 wherein the dose of the compound, or the pharmaceutically acceptable salt thereof, the first conjugate, or the pharmaceutically acceptable salt thereof, or the second conjugate, or the pharmaceutically acceptable salt thereof, is about 200 nmol/kg to about 500 nmol/kg of patient body weight.

34. The method of any one of clauses 1 to 33 wherein the dose of the compound, or the pharmaceutically acceptable salt thereof, the first conjugate, or the pharmaceutically acceptable salt thereof, or the second conjugate, or the pharmaceutically acceptable salt thereof, is about 250 nmol/kg to about 500 nmol/kg of patient body weight.

35. The method of any one of clauses 1 to 34 wherein the cancer is selected from lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, prostate cancer, chronic leukemia, acute leukemia, lymphocytic lymphoma, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, and adenocarcinoma of the gastroesophageal junction.

36. The method of any one of clauses 1 to 11 or 16 to 35 wherein the cancer is a folate receptor expressing cancer.

37. The method of clause 35 wherein the cancer is an endometrial cancer.

38. The method of clause 35 wherein the cancer is a non-small cell lung cancer.

39. The method of clause 35 wherein the cancer is an ovarian cancer.

40. The method of clause 35 wherein the cancer is a triple negative breast cancer.

41. The method of any one of clauses 1 to 40 wherein the CAR has a recognition region and the recognition region is a single chain fragment variable (scFv) region of an antibody.

42. The method of any one of clauses 1 to 11, 16 to 17, or 20 to 41 wherein the CAR has a recognition region and the recognition region of the CAR is a single chain fragment variable (scFv) region of an anti-FITC antibody.

43. The method of any one of clauses 1 to 42 wherein the CAR has a co-stimulation domain and the co-stimulation domain is selected from CD28, CD137 (4-1BB), CD134 (OX40), and CD278 (ICOS).

44. The method of any one of clauses 1 to 43 wherein the CAR has an activation signaling domain and the activation signaling domain is a T cell CD3ζ chain or an Fc receptor γ.

45. The method of any one of clauses 1 to 11, 16 to 17, or 20 to 41 wherein the CAR has a recognition region and the recognition region is a single chain fragment variable (scFv) region of an anti-FITC antibody, wherein the CAR has a co-stimulation domain and the co-stimulation domain is CD137 (4-1BB), and wherein the CAR has an activation signaling domain and the activation signaling domain is a T cell CD3ζ chain.

46. The method of any one of clauses 1 to 45 wherein multiple doses of the compound, or the pharmaceutically acceptable salt thereof, the first conjugate, or the pharmaceutically acceptable salt thereof, or the second conjugate, or the pharmaceutically acceptable salt thereof, and the CAR T cell composition are administered.

47. The method of any one of clauses 1 to 46 wherein the patient is imaged prior to administration of the compound, or the pharmaceutically acceptable salt thereof, the first conjugate, or the pharmaceutically acceptable salt thereof, or the second conjugate, or the pharmaceutically acceptable salt thereof, or prior to administration of the CAR T cell composition.

48. The method of any one of clauses 1 to 47 wherein the compound, or the pharmaceutically acceptable salt thereof, the first conjugate, or the pharmaceutically acceptable salt thereof, or the second conjugate, or the pharmaceutically acceptable salt thereof, is not an antibody, and does not comprise a fragment of an antibody.

49. The method of any one of clauses 1 to 48 wherein the targeting moiety is not a peptide epitope.

50. The method of any one of clauses 1 to 49 wherein cytokine release resulting in ‘off-target’ toxicity in the patient does not occur and wherein CAR T cell toxicity to the cancer occurs.

51. The method of any one of clauses 1 to 50 wherein ‘off-target’ tissue toxicity does not occur in the patient and wherein CAR T cell toxicity to the cancer occurs.

52. The method of any one of clauses 1 to 51 wherein the cancer comprises a tumor, wherein tumor size is reduced in the patient, and wherein ‘off-target’ toxicity does not occur.

53. A CAR T cell comprising a nucleic acid comprising SEQ ID NO:1.

54. A CAR T cell comprising a polypeptide comprising SEQ ID NO: 2.

55. An isolated nucleic acid comprising SEQ ID NO:1 and encoding a chimeric antigen receptor.

56. A chimeric antigen receptor polypeptide comprising SEQ ID NO: 2.

57. A vector comprising SEQ ID NO: 1.

58. The vector of clause 57 wherein the vector is a lentiviral vector.

59. The method, CAR T cell, isolated nucleic acid encoding a chimeric antigen receptor (CAR), or chimeric antigen receptor polypeptide of any one of clauses 1 to 56 wherein the CAR comprises human amino acid sequences.

60. The method, CAR T cell, isolated nucleic acid encoding a chimeric antigen receptor (CAR), or chimeric antigen receptor polypeptide of any one of clauses 1 to 56 wherein the CAR consists of human amino acid sequences.

61. A kit comprising at least two different types of bridges wherein the bridges comprise a small molecule ligand linked to a targeting moiety wherein the ligand in the at least two different types of bridges is different and wherein the ligand is selected from a folate, DUPA, a CAIX ligand, an NK-1R ligand, a ligand of gamma glutamyl transpeptidase, and a CCK2R ligand.

62. The kit of clause 61 wherein the ligand in at least one of the bridges is an NK-1R ligand.

63. The kit of clause 61 wherein the ligand in at least one of the bridges is a ligand of gamma glutamyl transpeptidase.

64. The kit of clause 61 wherein the ligand in at least one of the bridges is a folate.

65. The kit of any one of clauses 61 to 64 wherein the bridge has the formula

B-L-T,

wherein B represents the small molecule ligand, L represents the linker, and T represents the targeting moiety, and wherein L comprises a structure having the formula

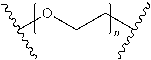

wherein n is an integer from 0 to 200.

66. The kit of clause 65 wherein n is an integer from 0 to 150.

67. The kit of clause 65 wherein n is an integer from 0 to 110.

68. The kit of clause 65 wherein n is an integer from 0 to 20.

69. The kit of clause 65 wherein n is an integer from 15 to 20.

70. The kit of clause 65 wherein n is an integer from 15 to 110.

71. The method of any one of clauses 1 to 10, 16 to 52, or 59 to 60, or the kit of any one of clauses 61 to 70 wherein the ligand is a CAIX ligand.

72. A conjugate of the formula

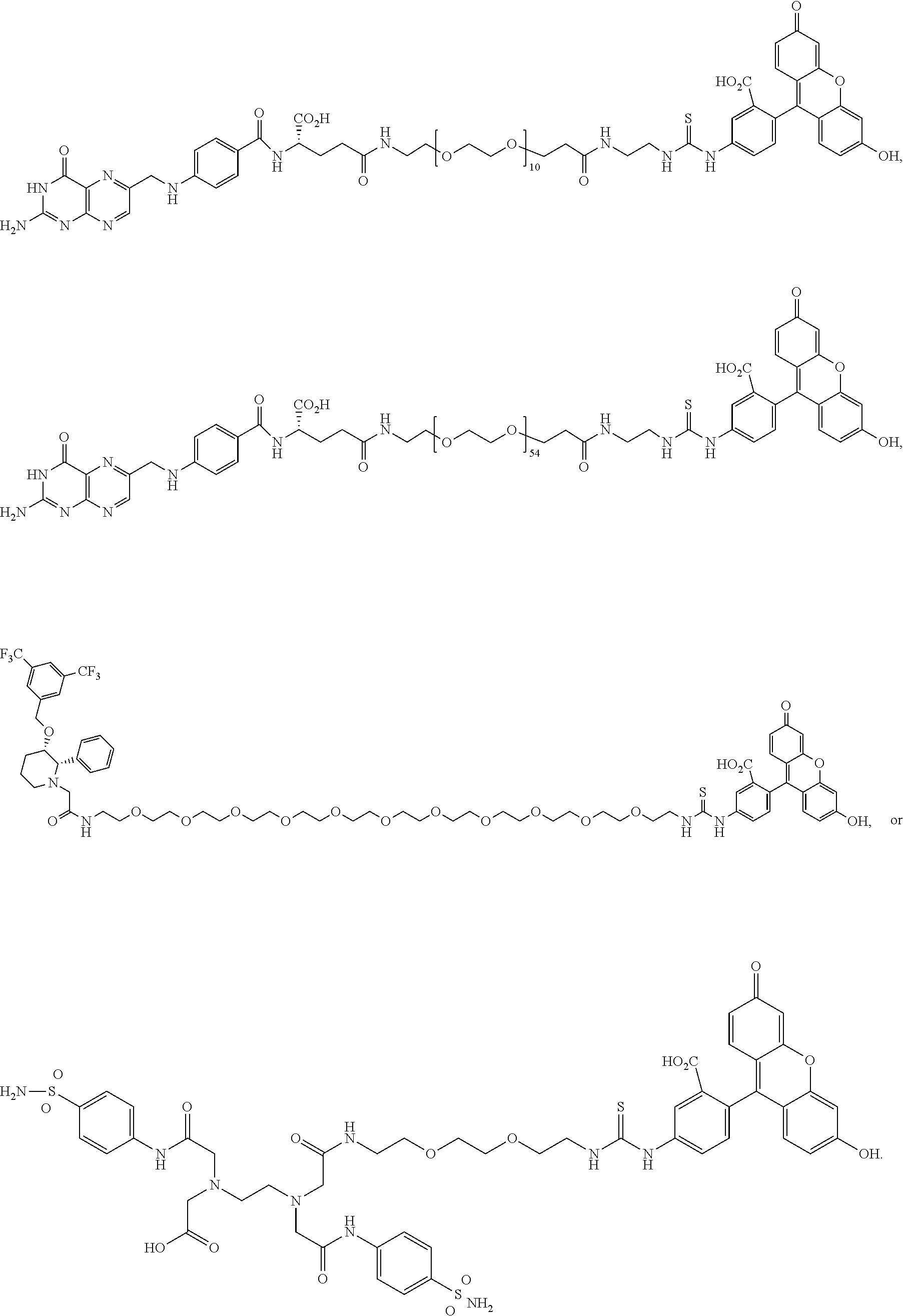

### BRIEF DESCRIPTION OF THE DRAWINGS

FIGS. 1A-B show CAR T cell proliferation using FITC-small molecule conjugates in different cell types with a (CAR T cell):target cell (cancer cell) ratio of 5:1. FIG. 1A shows CAR T cell proliferation in KB (FR+) cells. FIG. 1B shows CAR T cell proliferation in HEK293 (NK1R+) cells.

FIGS. 2A-F show inflammatory cytokine IFN-γ production by CAR T cells with FITC-small molecule conjugates in different cell types. FIG. 2A shows inflammatory cytokine IFN-γ production in KB (FR+) cells. FIG. 2B shows inflammatory cytokine IFN-γ production in LNCaP (PSMA+) cells. FIG. 2C shows inflammatory cytokine IFN-γ production in HEK293 (NK1R+) cells. FIG. 2D shows inflammatory cytokine IFN-γ production in KB (FR+) cells with different concentrations of FITC-Folate. FIG. 2E shows inflammatory cytokine IFN-γ production in KB (FR+) cells with different conjugates. FIG. 2F shows inflammatory cytokine IFN-γ production in KB (FR+) cells with different conjugates.

FIGS. 3A-F show in vitro toxicity of tumor cells treated with FITC-small molecule conjugates in different cell types. FIG. 3A shows in vitro toxicity in KB (FR+) cells. FIG. 3B shows in vitro toxicity in LNCaP (PSMA+) cells. FIG. 3C shows in vitro toxicity in HEK293 (NK1R+) cells. FIG. 3D shows in vitro toxicity in KB (FR+) cells as a function of different E:T (Effector cells:Target cells) ratios. FIG. 3E shows in vitro toxicity in KB (FR+) cells as a function of FITC-Folate concentration. FIG. 3F shows in vitro toxicity in KB (FR+) cells with different conjugates.

FIGS. 4A-B show activation of CAR T cells is correlated with the expression level of the tumor antigen on cancer cells. FIG. 4A shows tumor antigen FRα level. The highest peak is for KB (FR+) cells. FIG. 4B shows CAR T cell activation using FITC-small molecule conjugates as measured by IFN-γ production in MDA-MB-231 and KB cells.

FIGS. 5A-C show HEK293 (NK1R+) tumor xenografts and a CAR T cell therapy comprising treating CAR T cells with either a FITC-PEG11-NK1 conjugate or no conjugate. FIG. 5A shows tumor volume measured over 24 days.

FIG. 5B shows the body weight measured over 22 days of therapy. FIG. 5C shows the percentage of CAR T cells in CD3+ human T cells post CAR T cell injection along with FITC-PEG11-NK1.

FIGS. 6A-B show harvested organs from exemplary mice of the models used in FIGS. 5A-C. FIG. 6A shows harvested organs from the non-treatment group. FIG. 6B shows harvested organs after two weeks of CAR T cell therapy.

FIGS. 7A-C show MDA-MB-231 (FR+) xenografts under a CAR T cell therapy comprising treating the cells with CAR T cells with either a FITC-PEG12-Folate conjugate, a FITC-Folate conjugate, or no conjugate. FIG. 7A shows tumor volume measured over 23 days. FIG. 7B shows the body weight measured over 21 days of therapy. FIG. 7C shows the percentage of CAR T cells in CD3+ human T cells post CAR T cell injection.

FIGS. 8A-B show harvested organs from exemplary mice from the models shown in FIGS. 7A-C. FIG. 8A shows harvested organs from the non-treatment group. FIG. 8B shows harvested organs after three weeks of CAR T cell therapy comprising CAR T cells and the FITC-PEG12-Folate conjugate at 500 nmoles/kg body weight.

FIG. 9 shows blood indices of the HEK293 (NK1R+) xenograft model from FIGS. 5-6 and the MDA-MB-231 (FR+) xenograft model from FIGS. 7-8.

FIG. 10 shows differences in cytotoxicity towards KB (FR+) tumor cells treated with CAR T cells depending on the FITC-small molecule conjugate used.

FIG. 11 shows body weight percentage change in a KB tumor xenograft model using CAR T cells with different concentrations of a FITC-PEG-12-Folate conjugate.

FIGS. 12A-C show harvested organs from exemplary mice of the KB xenograft model shown in FIG. 11. FIG. 12A shows harvested organs from the non-treatment group. FIG. 12B shows harvested organs from the CAR T cell therapy group treated with 250 nmol/kg FITC-PEG-12-Folate. FIG. 12C shows harvested organs from the CAR T cell therapy group treated with CAR T cells and 500 nmol/kg FITC-PEG-12-Folate.

FIG. 13 shows blood indices of the mice from the KB xenograft model from FIGS. 11-12.

FIGS. 14A-B show the constructs used for CAR T transduction.

FIG. 14A shows the CAR4-1BBZ construct. FIG. 14B shows the lentiviral vector.

FIGS. 15A-B show flow cytometry analysis of transduced T cells.

FIG. 15A shows the non-transduced cells. FIG. 15B shows the transduced cells.

FIGS. 16A-B show fluorescent microscopy of transduced CAR T cells. FIG. 16A shows GFP imaging indicating transduction. FIG. 16B shows FITC folate localizing to the positively transduced cells.

FIG. 17 shows activation of CAR T cells as measured by relative expression of CD69 as a function of the conjugate used.

FIG. 18 shows tumor heterogeneity of KB, LNCaP, and CAR T cells as a function of the conjugate used.

FIGS. 19A-C shows anti-tumor efficacy when the same anti-FITC CAR T cell (10⁷ cells) was introduced to mice bearing two different tumors arising from two different cell lines (i.e. MDA-MB-231(FR+) and HEK (NK1R+)) on separate flanks, after which either PBS only (FIG. 19A), FITC-PEG11-NK1R (500 nmole/kg) (FIG. 19B), or FITC-PEG11-NK1R (500 nmole/kg) plus FITC-PEG12-Folate (500 nmole/kg) (FIG. 19C) was injected every other day. FIG. 19A: (●) FR+(MDA-MB-231): CAR T cell+PBS, (▪) NKIR+(HEK): CAR T cell+PBS; FIG. 19B: (●) FR+(MDA-MB-231): CAR T cell+PBS, (▪) NKIR+(HEK): CAR T cell+FITC-PEG11-NKIR (500 nmole/kg); FIG. 19C: (●) FR+(MDA-MB-231): CAR T cell+FITC-PEG12-FA (500 nmole/kg), (▪) NKIR+(HEK): CAR T cell+PBS.

### DEFINITIONS

As used herein, “a” or “an” may mean one or more. As used herein, “about” in reference to a numeric value, including, for example, whole numbers, fractions, and percentages, generally refers to a range of numerical values (e.g., +/−5% to 10% of the recited value) that one of ordinary skill in the art would consider equivalent to the recited value (e.g., having the same function or result).

As used herein, the terms “treat,” “treating,” “treated,” or “treatment” refer to both therapeutic treatment and prophylactic or preventative treatment.

As used herein, the terms “ameliorate,” “ameliorating,” “amelioration,” or “ameliorated” in reference to cancer can mean reducing the symptoms of the cancer, reducing the size of a tumor, completely or partially removing the tumor (e.g., a complete or partial response), causing stable disease, preventing progression of the cancer (e.g., progression free survival), or any other effect on the cancer that would be considered by a physician to be a therapeutic or preventative treatment of the cancer.

As used herein, the terms “administer,” administering,” or “administered” mean all means of introducing the compound, or pharmaceutically acceptable salt thereof, the first conjugate, or pharmaceutically acceptable salt thereof, the second conjugate, or pharmaceutically acceptable salt thereof, or CAR T cell composition described herein to the patient, including, but not limited to, oral (po), intravenous (iv), intramuscular (im), subcutaneous (sc), and transdermal.

As used herein, the term “off-target toxicity” means organ damage or a reduction in the patient's weight that is unacceptable to the physician treating the patient, or any other effect that is unacceptable to the physician treating the patient, such as B cell aplasia.

As used herein, the terms “transduction” and “transfection” are used equivalently and the terms mean introducing a nucleic acid into a cell by any artificial method, including viral and non-viral methods.

### DETAILED DESCRIPTION OF THE ILLUSTRATIVE EMBODIMENTS

In the various embodiments described herein, a small molecule ligand linked to a targeting moiety by a linker is used as a bridge between a cancer and CAR T cells (i.e, cytotoxic T cells expressing a chimeric antigen receptor). The bridge directs the CAR T cells to the cancer for amelioration of the cancer. In one embodiment, the “small molecule ligand” can be a folate, a CAIX ligand, DUPA, an NK-1R ligand, a ligand of gamma glutamyl transpeptidase, or a CCK2R ligand, each of which is a small molecule ligand that binds specifically to a cancer cell type (i.e., the receptor for each of these ligands is overexpressed on cancers compared to normal tissues).

The “targeting moiety” linked to the small molecule ligand binds to the recognition region of the genetically engineered CAR expressed by CAR T cells. Accordingly, the recognition region of the CAR (e.g., a single chain fragment variable region (scFv) of an antibody) is directed to the “targeted moiety.” Thus, the small molecule ligand linked to a targeting moiety by a linker acts as a bridge between the cancer and the CAR T cells directing the CAR T cells to the cancer for amelioration of the cancer. In various embodiments, the bridge between the cancer and the CAR T cells can be any of the conjugates shown in Examples 5 to 12.

The bridge is a small organic molecule so clearance from the bloodstream can be rapidly achieved (e.g., about 20 minutes or less). In one aspect, the CAR T cell response can be targeted to only those cancer cells expressing a receptor for the small molecule ligand portion of the ‘bridge,’ thereby reducing off-target toxicity to normal tissues. In another aspect, CAR T cell activation can be controlled due to the rapid clearance of the bridge from the bloodstream and to the ability to vary the dose and structure of the bridge to regulate CAR T cell activation. Additionally, this system can be ‘universal’ because one type of CAR T cell construct can be used to target a wide variety of cancers. Illustratively, the targeting moiety recognized by the CAR T cell may remain constant so that one type of CAR T cell construct can be used, while the small molecule ligand that binds to the cancer is altered to allow targeting of a wide variety of cancers.

In one embodiment, the inventors have discovered that varying the dose of the small molecule ligand linked to a targeting moiety by a linker (i.e., the bridge), can result in the ability to control CRS in vivo upon CAR T cell activation. In another embodiment, the inventors have discovered that varying the linker in the small molecule ligand linked to a targeting moiety (the bridge) can control CRS in vivo upon CAR T cell activation. In yet another embodiment, combinations of these methods can be used for precise control of CAR T cell activation and cytokine release in vivo.

In various embodiments described in the clause list below and in the claims, the small molecule ligand linked to a targeting moiety by a linker is referred to as a “compound,” a “first conjugate,” or a “second conjugate.” The term “compound” is used in embodiments where the dose of the small molecule ligand linked to a targeting moiety by a linker is varied to control cytokine release in vivo. The terms “first conjugate” and “second conjugate” are used in embodiments where two different conjugates are administered to a patient. For example, the linker in the small molecule ligand linked to a targeting moiety can be varied to control cytokine release in vivo, or the conjugates can be modified to contain different small molecule ligands or different targeting moieties.

Several embodiments are described by the following enumerated clauses:

1. A method of treatment of a cancer, the method comprising
i) administering to a patient a first dose of a compound, or a pharmaceutically acceptable salt thereof, wherein the compound comprises a small molecule ligand linked to a targeting moiety by a linker;
ii) administering to the patient a CAR T cell composition wherein the CAR T cell comprises a CAR directed to the targeting moiety;
ii) administering to the patient a second dose of the compound, or the pharmaceutically acceptable salt thereof, wherein the second dose is different than the first dose; and
iv) treating the patient to ameliorate the cancer.

2. A method of treatment of a cancer, the method comprising
i) administering to the patient a first conjugate, or a pharmaceutically acceptable salt thereof;
ii) administering to the patient a CAR T cell composition wherein the CAR T cell comprises a CAR directed to the targeting moiety; iii) administering to the patient a second conjugate, or a pharmaceutically acceptable salt thereof, wherein the first and the second conjugate each comprise a small molecule ligand linked to a targeting moiety by a linker and wherein the first conjugate and the second conjugate are different; and
iv) treating the patient to ameliorate the cancer.

3. A method of treatment of a cancer, the method comprising
i) administering to a patient a first dose of a first conjugate, or a pharmaceutically acceptable salt thereof;
ii) administering to the patient a CAR T cell composition wherein the CAR T cell comprises a CAR directed to the targeting moiety;
ii) administering to the patient a second dose of a second conjugate, or a pharmaceutically acceptable salt thereof, wherein the first conjugate and the second conjugate each comprise a small molecule ligand linked to a targeting moiety, wherein the first conjugate and the second conjugate are different, and wherein the first dose and the second dose are different; and
iv) treating the patient to ameliorate the cancer.

4. The method of clause 2 or 3 wherein the linker in the first conjugate, or the pharmaceutically acceptable salt thereof, and the linker in the second conjugate, or the pharmaceutically acceptable salt thereof, are different.

5. The method of clause 2 or 3 wherein the linker in the first conjugate, or the pharmaceutically acceptable salt thereof, and the linker in the second conjugate, or the pharmaceutically acceptable salt thereof, are the same.

6. The method of any one of clauses 2 to 5 wherein the ligand in the first conjugate, or the pharmaceutically acceptable salt thereof, and the ligand in the second conjugate, or the pharmaceutically acceptable salt thereof, are different.

7. The method of any one of clauses 2 to 5 wherein the ligand in the first conjugate, or the pharmaceutically acceptable salt thereof, and the ligand in the second conjugate, or the pharmaceutically acceptable salt thereof, are the same.

8. The method of any one of clauses 2 to 7 wherein the targeting moiety in the first conjugate, or the pharmaceutically acceptable salt thereof, and the targeting moiety in the second conjugate, or the pharmaceutically acceptable salt thereof, are different.

9. The method of any one of clauses 2 to 7 wherein the targeting moiety in the first conjugate, or the pharmaceutically acceptable salt thereof, and the targeting moiety in the second conjugate, or the pharmaceutically acceptable salt thereof, are the same.

10. The method of any one of clauses 1 to 9 wherein the ligand is selected from a folate, DUPA, a CAIX ligand, an NK-1R ligand, a ligand of gamma glutamyl transpeptidase, and a CCK2R ligand.

11. The method of clause 10 wherein the ligand is a folate.

12. The method of clause 10 wherein the ligand is an NK-1R ligand.

13. The method of clause 10 wherein the ligand is DUPA.

14. The method of clause 10 wherein the ligand is a CCK2R ligand.

15. The method of clause 10 wherein the ligand is a ligand of gamma glutamyl transpeptidase.

16. The method of any one of clauses 1 to 15 wherein the targeting moiety is selected from 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester (PFP), tetrafluorophenyl ester (TFP), a knottin, a centyrin, and a DARPin.

17. The method of clause 16 wherein the targeting moiety is FITC.

18. The method of clause 16 wherein the targeting moiety is DNP.

19. The method of clause 16 wherein the targeting moiety is TNP.

20. The method of any one of clauses 1 to 19 wherein the linker comprises polyethylene glycol (PEG), polyproline, a hydrophilic amino acid, a sugar, an unnatural peptidoglycan, a polyvinylpyrrolidone, and/or pluronic F-127.

21. The method of clause 20 wherein the linker comprises PEG.

22. The method of any one of clauses 1 to 21 wherein the compound, or the pharmaceutically acceptable salt thereof, the first conjugate, or the pharmaceutically acceptable salt thereof, or the second conjugate or the pharmaceutically acceptable salt thereof, has the formula

B-L-T,

wherein B represents the small molecule ligand, L represents the linker, and T represents the targeting moiety, and wherein L comprises a structure having the formula

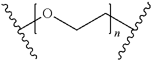

wherein n is an integer from 0 to 200.

23. The method of clause 22 wherein n is an integer from 0 to 150.

24. The method of clause 22 wherein n is an integer from 0 to 110.

25. The method of clause 22 wherein n is an integer from 0 to 20.

26. The method of clause 22 wherein n is an integer from 15 to 20.

27. The method of clause 22 wherein n is an integer from 15 to 110.

28. The method of any one of clauses 1 to 27 wherein the linker comprises PEG and the targeting moiety is FITC, or a pharmaceutically acceptable salt thereof.

29. The method of any one of clauses 1 to 28 wherein the dose of the compound, or the pharmaceutically acceptable salt thereof, the first conjugate, or the pharmaceutically acceptable salt thereof, or the second conjugate, or the pharmaceutically acceptable salt thereof, is about 10 nmol/kg to about 3000 nmol/kg of patient body weight.

30. The method of any one of clauses 1 to 29 wherein the dose of the compound, or the pharmaceutically acceptable salt thereof, the first conjugate, or the pharmaceutically acceptable salt thereof, or the second conjugate, or the pharmaceutically acceptable salt thereof, is about 50 nmol/kg to about 2000 nmol/kg of patient body weight.

31. The method of any one of clauses 1 to 30 wherein the dose of the compound, or the pharmaceutically acceptable salt thereof, the first conjugate, or the pharmaceutically acceptable salt thereof, or the second conjugate, or the pharmaceutically acceptable salt thereof, is about 100 nmol/kg to about 1000 nmol/kg of patient body weight.

32. The method of any one of clauses 1 to 31 wherein the dose of the compound, or the pharmaceutically acceptable salt thereof, the first conjugate, or the pharmaceutically acceptable salt thereof, or the second conjugate, or the pharmaceutically acceptable salt thereof, is about 100 nmol/kg to about 600 nmol/kg of patient body weight.

33. The method of any one of clauses 1 to 32 wherein the dose of the compound, or the pharmaceutically acceptable salt thereof, the first conjugate, or the pharmaceutically acceptable salt thereof, or the second conjugate, or the pharmaceutically acceptable salt thereof, is about 200 nmol/kg to about 500 nmol/kg of patient body weight.

34. The method of any one of clauses 1 to 33 wherein the dose of the compound, or the pharmaceutically acceptable salt thereof, the first conjugate, or the pharmaceutically acceptable salt thereof, or the second conjugate, or the pharmaceutically acceptable salt thereof, is about 250 nmol/kg to about 500 nmol/kg of patient body weight.

35. The method of any one of clauses 1 to 34 wherein the cancer is selected from lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, prostate cancer, chronic leukemia, acute leukemia, lymphocytic lymphoma, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, and adenocarcinoma of the gastroesophageal junction.

36. The method of any one of clauses 1 to 11 or 16 to 35 wherein the cancer is a folate receptor expressing cancer.

37. The method of clause 35 wherein the cancer is an endometrial cancer.

38. The method of clause 35 wherein the cancer is a non-small cell lung cancer.

39. The method of clause 35 wherein the cancer is an ovarian cancer.

40. The method of clause 35 wherein the cancer is a triple negative breast cancer.

41. The method of any one of clauses 1 to 40 wherein the CAR has a recognition region and the recognition region is a single chain fragment variable (scFv) region of an antibody.

42. The method of any one of clauses 1 to 11, 16 to 17, or 20 to 41 wherein the CAR has a recognition region and the recognition region of the CAR is a single chain fragment variable (scFv) region of an anti-FITC antibody.

43. The method of any one of clauses 1 to 42 wherein the CAR has a co-stimulation domain and the co-stimulation domain is selected from CD28, CD137 (4-1BB), CD134 (OX40), and CD278 (ICOS).

44. The method of any one of clauses 1 to 43 wherein the CAR has an activation signaling domain and the activation signaling domain is a T cell CD3ζ chain or an Fc receptor γ.

45. The method of any one of clauses 1 to 11, 16 to 17, or 20 to 41 wherein the CAR has a recognition region and the recognition region is a single chain fragment variable (scFv) region of an anti-FITC antibody, wherein the CAR has a co-stimulation domain and the co-stimulation domain is CD137 (4-1BB), and wherein the CAR has an activation signaling domain and the activation signaling domain is a T cell CD3 chain.

46. The method of any one of clauses 1 to 45 wherein multiple doses of the compound, or the pharmaceutically acceptable salt thereof, the first conjugate, or the pharmaceutically acceptable salt thereof, or the second conjugate, or the pharmaceutically acceptable salt thereof, and the CAR T cell composition are administered.

47. The method of any one of clauses 1 to 46 wherein the patient is imaged prior to administration of the compound, or the pharmaceutically acceptable salt thereof, the first conjugate, or the pharmaceutically acceptable salt thereof, or the second conjugate, or the pharmaceutically acceptable salt thereof, or prior to administration of the CAR T cell composition.

48. The method of any one of clauses 1 to 47 wherein the compound, or the pharmaceutically acceptable salt thereof, the first conjugate, or the pharmaceutically acceptable salt thereof, or the second conjugate, or the pharmaceutically acceptable salt thereof, is not an antibody, and does not comprise a fragment of an antibody.

49. The method of any one of clauses 1 to 48 wherein the targeting moiety is not a peptide epitope.

50. The method of any one of clauses 1 to 49 wherein cytokine release resulting in ‘off-target’ toxicity in the patient does not occur and wherein CAR T cell toxicity to the cancer occurs.

51. The method of any one of clauses 1 to 50 wherein ‘off-target’ tissue toxicity does not occur in the patient and wherein CAR T cell toxicity to the cancer occurs.

52. The method of any one of clauses 1 to 51 wherein the cancer comprises a tumor, wherein tumor size is reduced in the patient, and wherein ‘off-target’ toxicity does not occur.

53. A CAR T cell comprising a nucleic acid comprising SEQ ID NO:1.

54. A CAR T cell comprising a polypeptide comprising SEQ ID NO: 2.

55. An isolated nucleic acid comprising SEQ ID NO:1 and encoding a chimeric antigen receptor.

56. A chimeric antigen receptor polypeptide comprising SEQ ID NO: 2.

57. A vector comprising SEQ ID NO: 1.

58. The vector of clause 57 wherein the vector is a lentiviral vector.

59. The method, CAR T cell, isolated nucleic acid encoding a chimeric antigen receptor (CAR), or chimeric antigen receptor polypeptide of any one of clauses 1 to 56 wherein the CAR comprises human amino acid sequences.

60. The method, CAR T cell, isolated nucleic acid encoding a chimeric antigen receptor (CAR), or chimeric antigen receptor polypeptide of any one of clauses 1 to 56 wherein the CAR consists of human amino acid sequences.

61. A kit comprising at least two different types of bridges wherein the bridges comprise a small molecule ligand linked to a targeting moiety wherein the ligand in the at least two different types of bridges is different and wherein the ligand is selected from a folate, DUPA, a CAIX ligand, an NK-1R ligand, a ligand of gamma glutamyl transpeptidase, and a CCK2R ligand.

62. The kit of clause 61 wherein the ligand in at least one of the bridges is an NK-1R ligand.

63. The kit of clause 61 wherein the ligand in at least one of the bridges is a ligand of gamma glutamyl transpeptidase.

64. The kit of clause 61 wherein the ligand in at least one of the bridges is a folate.

65. The kit of any one of clauses 61 to 64 wherein the bridge has the formula

B-L-T,

wherein B represents the small molecule ligand, L represents the linker, and T represents the targeting moiety, and wherein L comprises a structure having the formula

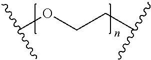

wherein n is an integer from 0 to 200.

66. The kit of clause 65 wherein n is an integer from 0 to 150.

67. The kit of clause 65 wherein n is an integer from 0 to 110.

68. The kit of clause 65 wherein n is an integer from 0 to 20.

69. The kit of clause 65 wherein n is an integer from 15 to 20.

70. The kit of clause 65 wherein n is an integer from 15 to 110.

71. The method of any one of clauses 1 to 10, 16 to 52, or 59 to 60, or the kit of any one of clauses 61 to 70 wherein the ligand is a CAIX ligand.

72. A conjugate of the formula

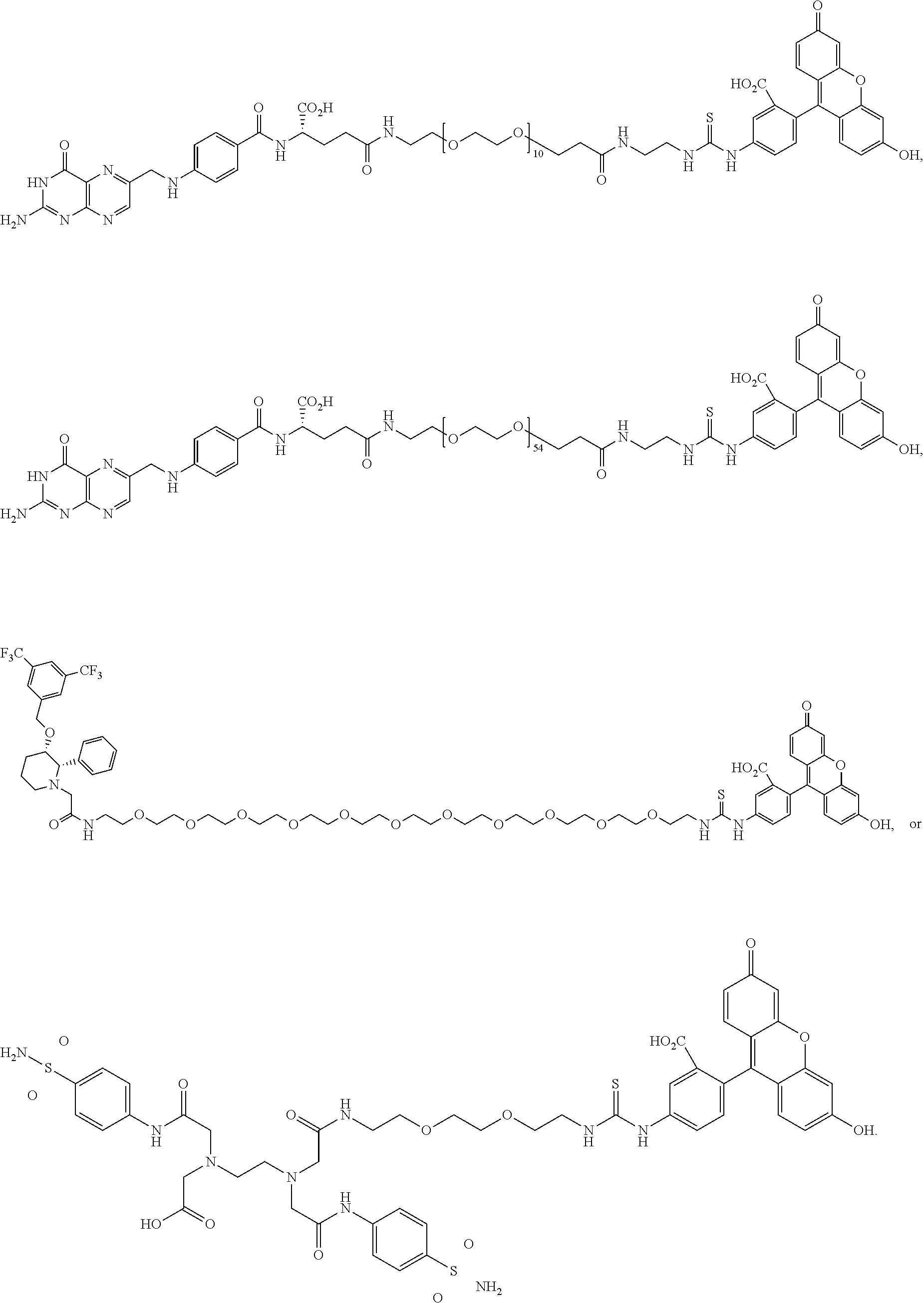

As described herein a “patient” can be a human or in the case of veterinary applications, the patient can be a laboratory, an agricultural, a domestic, or a wild animal. In one aspect, the patient can be a laboratory animal such as a rodent (e.g., mouse, rat, hamster, etc.), a rabbit, a monkey, a chimpanzee, a domestic animal such as a dog, a cat, or a rabbit, an agricultural animal such as a cow, a horse, a pig, a sheep, a goat, or a wild animal in captivity such as a bear, a panda, a lion, a tiger, a leopard, an elephant, a zebra, a giraffe, a gorilla, a dolphin, or a whale. In the methods described herein, the step of “treating the patient to ameliorate the cancer” can comprise or consist of the administering steps in the method.

In one illustrative embodiment, the small molecule ligand linked to a targeting moiety by a linker (the bridge) comprises fluorescein isothiocyanate (FITC) linked to the small molecule ligand. The cancer overexpresses a receptor for the small molecule ligand. As a second component, for example, cytotoxic T cells are transformed to express a CAR that comprises anti-FITC scFv. In this aspect, the CAR targets FITC decorating the cancer with FITC molecules as a result of binding of the small molecule ligand to the cancer. Thus, toxicity to normal, non-target cells can be avoided. When the anti-FITC CAR-expressing T cells bind FITC, the CAR T cells are activated and the cancer is ameliorated (e.g., by killing the cancer cells).

In one embodiment, the “small molecule ligand” can be a folate, DUPA (a ligand bound by PSMA-positive human prostate cancer cells and other cancer cell types), an NK-1R ligand (receptors for NK-1R the ligand found, for example, on cancers of the colon and pancreas), a CAIX ligand (receptors for the CAIX ligand found, for example, on renal, ovarian, vulvar, and breast cancers), a ligand of gamma glutamyl transpeptidase (the transpeptidase overexpressed, for example, in ovarian cancer, colon cancer, liver cancer, astrocytic gliomas, melanomas, and leukemias), or a CCK2R ligand (receptors for the CCK2R ligand found on cancers of the thyroid, lung, pancreas, ovary, brain, stomach, gastrointestinal stroma, and colon, among others), each of which is a small molecule ligand that binds specifically to a cancer cell type (i.e., the receptor for each of these ligands can be overexpressed on cancers compared to normal tissues). In one embodiment, a DUPA derivative can be the ligand of the small molecule ligand linked to a targeting moiety, and DUPA derivatives are described in WO 2015/057852, incorporated herein by reference.

In one embodiment, the small molecule ligand is a folate. The folate can be folic acid, a folic acid analog, or another folate receptor-binding molecule. In various embodiments, analogs of folate that can be used include folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refers to the art recognized analogs having a carbon atom substituted for one or two nitrogen atoms in the naturally occurring folic acid structure. For example, the deaza analogs include the 1-deaza, 3-deaza, 5-deaza, 8-deaza, and 10-deaza analogs. The dideaza analogs include, for example, 1,5 dideaza, 5,10-dideaza, 8,10-dideaza, and 5,8-dideaza analogs. The foregoing folic acid analogs are conventionally termed “folates,” reflecting their capacity to bind to folate receptors. Other folate receptor-binding analogs include aminopterin, amethopterin (methotrexate), N10-methylfolate, 2-deamino-hydroxyfolate, deaza analogs such as 1-deazamethopterin or 3-deazamethopterin, and 3′,5′-dichloro-4-amino-4-deoxy-N10-methylpteroylglutamic acid (dichloromethotrexate).

In one embodiment, the small molecule ligand may have a mass of less than about 10,000 Daltons, less than about 9000 Daltons, less than about 8,000 Daltons, less than about 7000 Daltons, less than about 6000 Daltons, less than about 5000 Daltons, less than about 4500 Daltons, less than about 4000 Daltons, less than about 3500 Daltons, less than about 3000 Daltons, less than about 2500 Daltons, less than about 2000 Daltons, less than about 1500 Daltons, less than about 1000 Daltons, or less than about 500 Daltons. In another embodiment, the small molecule ligand may have a mass of about 1 to about 10,000 Daltons, about 1 to about 9000 Daltons, about 1 to about 8,000 Daltons, about 1 to about 7000 Daltons, about 1 to about 6000 Daltons, about 1 to about 5000 Daltons, about 1 to about 4500 Daltons, about 1 to about 4000 Daltons, about 1 to about 3500 Daltons, about 1 to about 3000 Daltons, about 1 to about 2500 Daltons, about 1 to about 2000 Daltons, about 1 to about 1500 Daltons, about 1 to about 1000 Daltons, or about 1 to about 500 Daltons.

In one aspect, the “targeting moiety” that binds to the CAR expressed by CAR T cells can be selected, for example, from 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester (PFP), tetrafluorophenyl ester (TFP), a knottin, a centyrin, and a DARPin. The identity of the targeting moiety is limited only in that it should be recognized and bound by the CAR, preferably with specificity, and that it have a relatively low molecular weight. In various aspects, exemplary targeting moieties are haptens that include small molecular weight organic molecules.

In one illustrative embodiment, the targeting moiety can have the following illustrative structure:

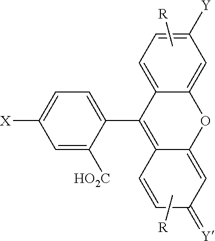

where X is oxygen, nitrogen, or sulfur, and where X is attached to linker L; Y is OR^{a}, NR^{a}₂, or NR^{a}₃⁺; and Y′ is O, NR^{a}, or NR^{a}₂⁺; where each R is independently selected in each instance from H, fluoro, sulfonic acid, sulfonate, and salts thereof, and the like; and Ra is hydrogen or alkyl.

In one illustrative aspect, the linker in the compound, or pharmaceutically acceptable salt thereof, the first conjugate, or pharmaceutically acceptable salt thereof, the second conjugate, or pharmaceutically acceptable salt thereof, described herein can be a direct linkage (e.g., a reaction between the isothiocyanate group of FITC and a free amine group of a small molecule ligand) or the linkage can be through an intermediary linker. In one embodiment, if present, an intermediary linker can be any biocompatible linker known in the art, such as a divalent linker. In one illustrative embodiment, the divalent linker can comprise about 1 to about 30 carbon atoms. In another illustrative embodiment, the divalent linker can comprise about 2 to about 20 carbon atoms. In other embodiments, lower molecular weight divalent linkers (i.e., those having an approximate molecular weight of about 30 to about 300) are employed. In another embodiment, linkers lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In various embodiments, the small molecule ligand linked to a targeting moiety can be of the formula

B-L-T,

wherein B represents the small molecule ligand, L represents the linker, and T represents the targeting moiety, and wherein L comprises a structure having the formula

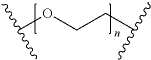

wherein n is an integer from 0 to 200. In another embodiment, n can be an integer from 0 to 150, 0 to 110, 0 to 100, 0 to 90, 0 to 80, 0 to 70, 0 to 60, 0 to 50, 0 to 40, 0 to 30, 0 to 20, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, 0 to 1, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 15 to 31, 15 to 32, 15 to 33, 15 to 34, 15 to 35, 15 to 36, 15 to 37, 15 to 38, 15 to 39, 15 to 40, 15 to 50, 15 to 60, 15 to 70, 15 to 80, 15 to 90, 15 to 100, 15 to 110, 15 to 120, 15 to 130, 15 to 140, 15 to 150, or n can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 108, 110, 120, 130, 140, or 150.

In another embodiment, the linker may be a divalent linker that may include one or more spacers. Illustrative spacers are shown in the following table. The following non-limiting, illustrative spacers are described where * indicates the point of attachment to the small molecule ligand or the targeting moiety.

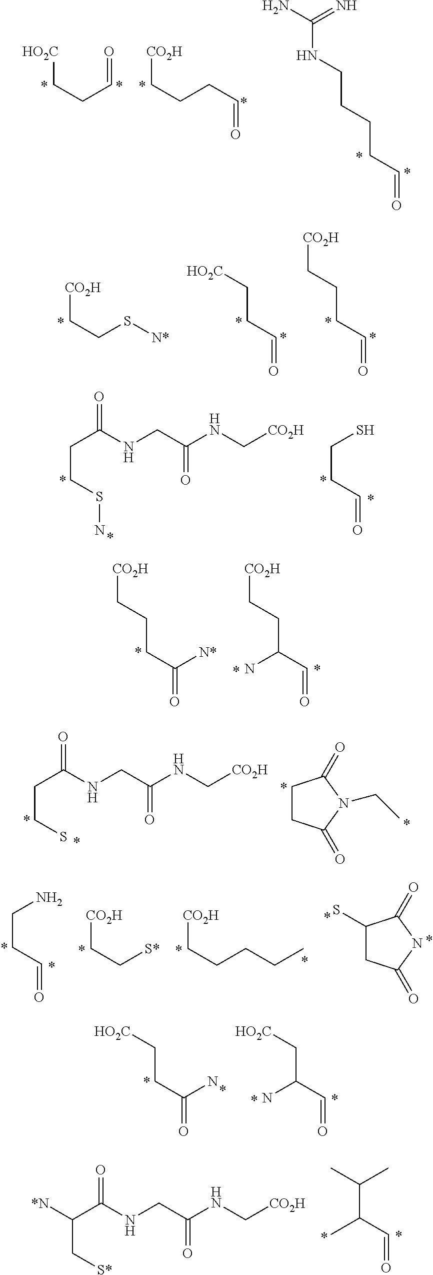

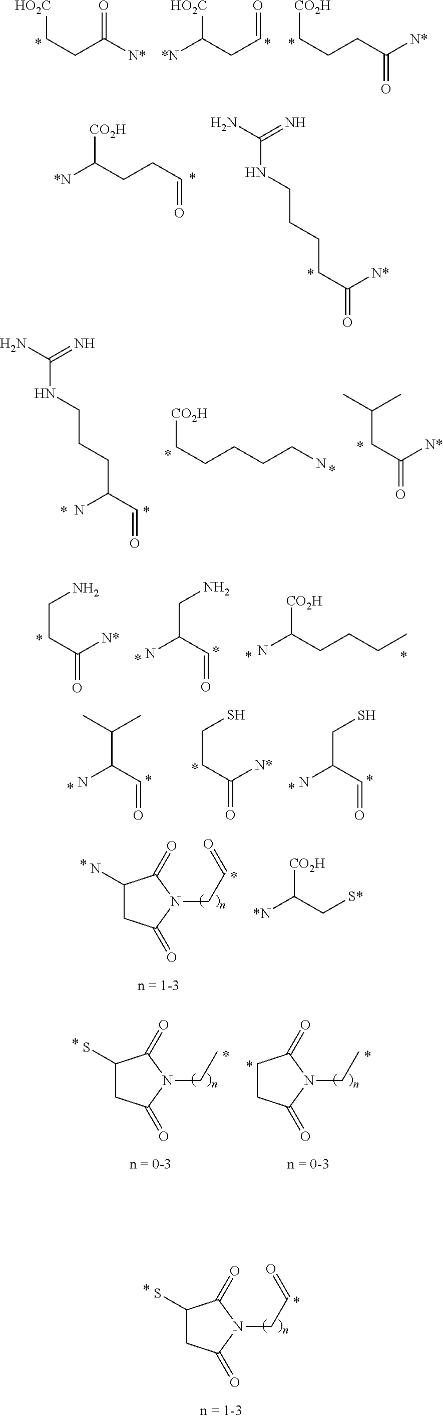

In other embodiments, the small molecule ligand linked to a targeting moiety (bridge) can have any of the following structures.

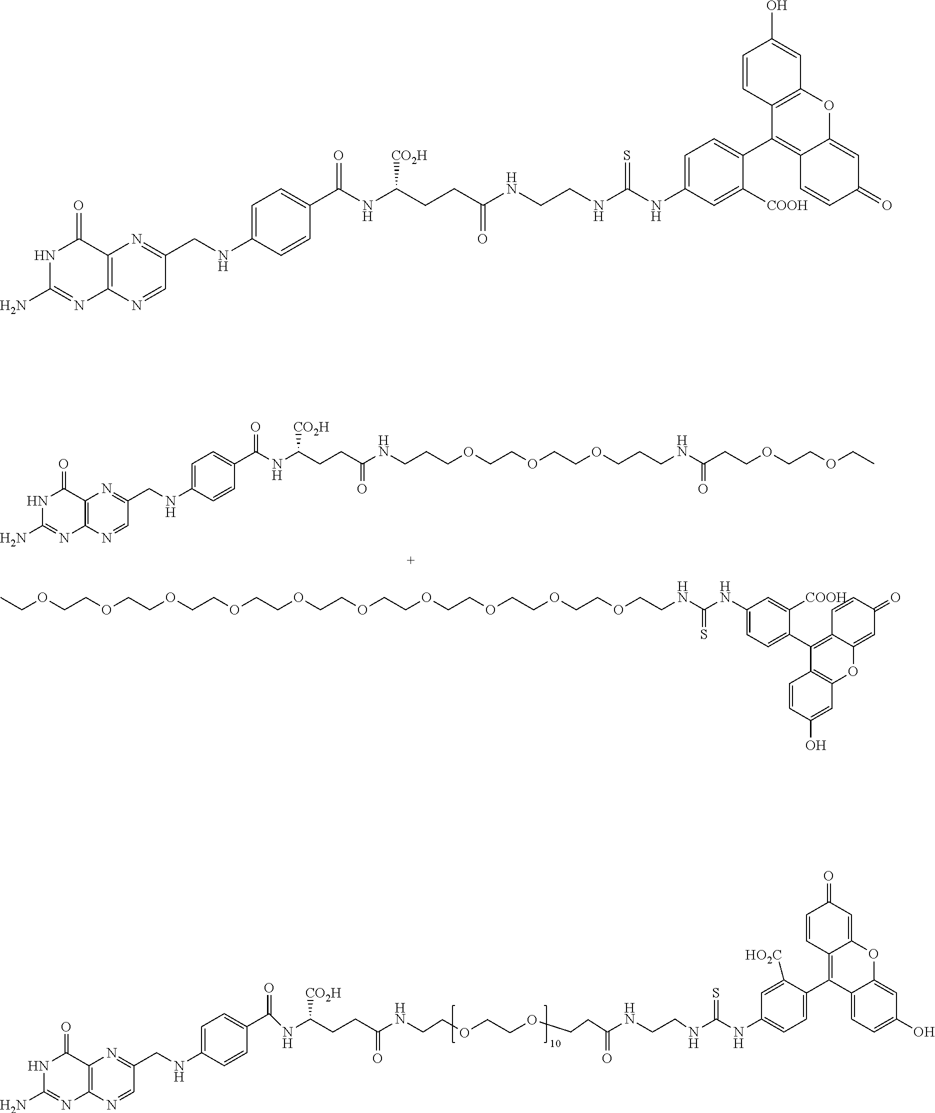

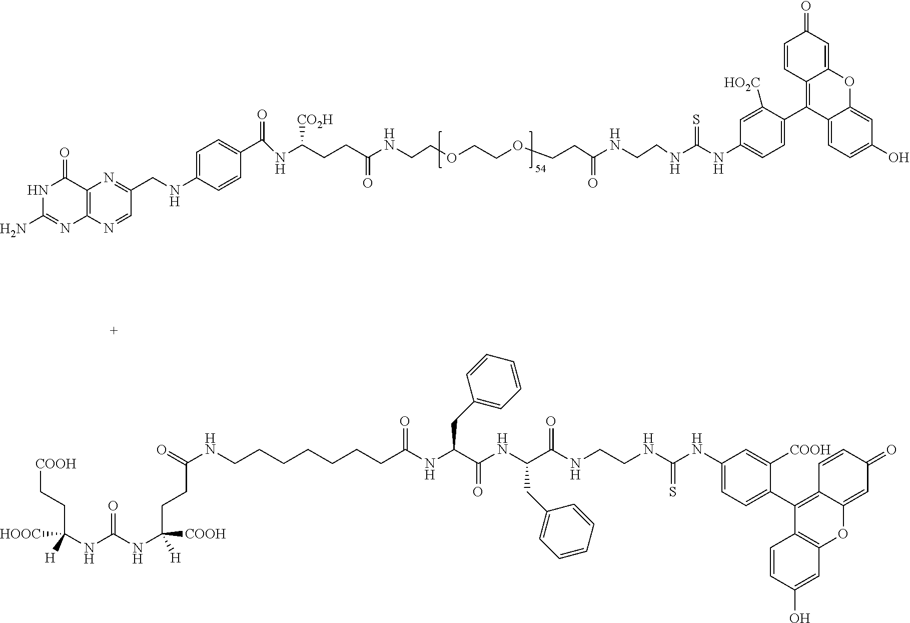

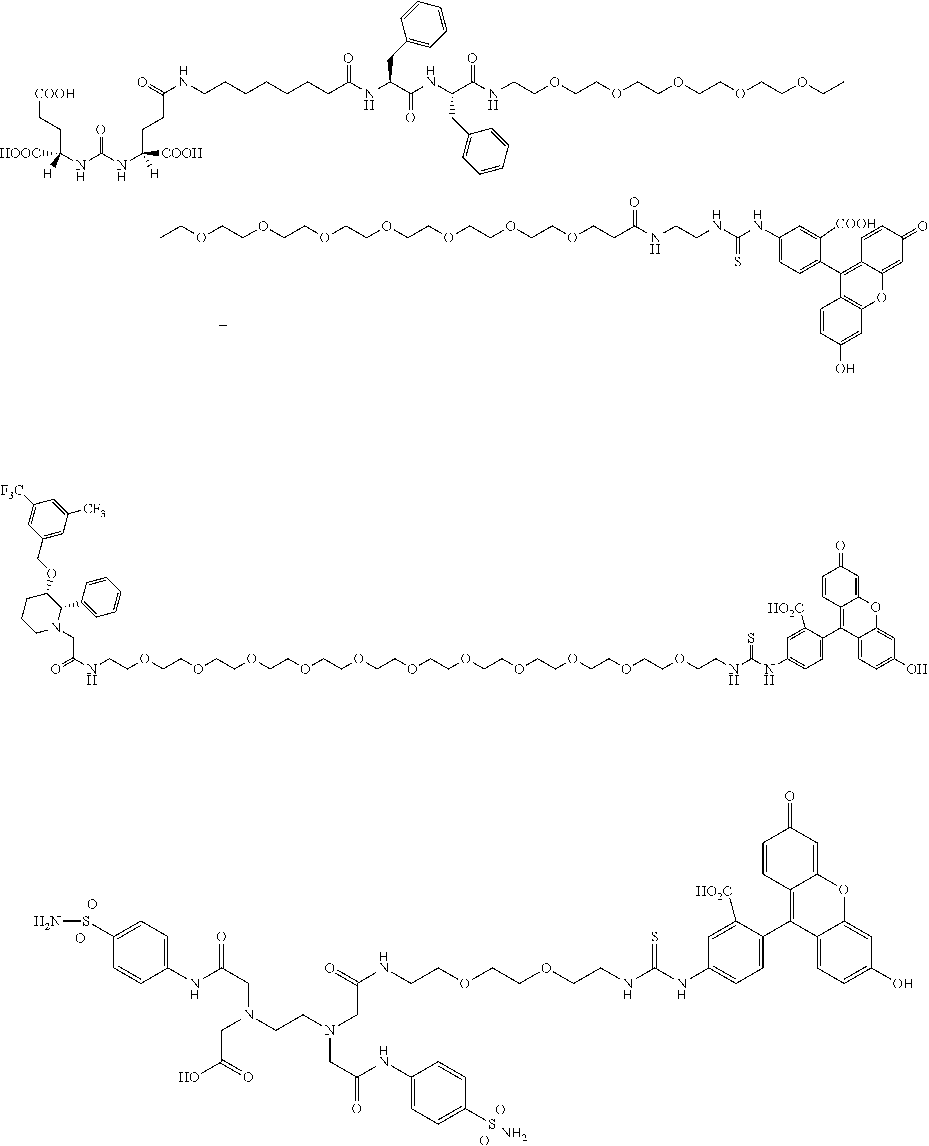

In other embodiments, the compound, or the pharmaceutically acceptable salt thereof, the first conjugate, or the pharmaceutically acceptable salt thereof, or the second conjugate, or the pharmaceutically acceptable salt thereof, is not an antibody, and does not comprise a fragment of an antibody. In yet another embodiment, the targeting moiety is not a peptide epitope.

In one illustrative aspect, different type of conjugates (e.g., a first conjugate and a second conjugate) can be administered to the patient. For example, the linker in the first conjugate, or the pharmaceutically acceptable salt thereof, and the linker in the second conjugate, or the pharmaceutically acceptable salt thereof, can be the same or different. In another aspect, the ligand in the first conjugate, or the pharmaceutically acceptable salt thereof, and the ligand in the second conjugate, or the pharmaceutically acceptable salt thereof, can be the same or different. In another exemplary embodiment, the targeting moiety in the first conjugate, or the pharmaceutically acceptable salt thereof, and the targeting moiety in the second conjugate, or the pharmaceutically acceptable salt thereof, can be the same or different. Any combinations of these embodiments are also contemplated along with any combinations of the doses described below.

In still another embodiment, a kit is provided comprising at least two different types of bridges, wherein the bridges comprise a small molecule ligand linked to a targeting moiety wherein the ligand in the at least two different types of bridges is different and wherein the ligand is selected from a folate, DUPA, a CAIX ligand, an NK-1R ligand, a ligand of gamma glutamyl transpeptidase, and a CCK2R ligand. In this embodiment, the ligand in at least one of the bridges can be an NK-1R ligand, a ligand of gamma glutamyl transpeptidase, a folate, a CAIX ligand, a CCK2R ligand, or DUPA.

In another aspect, the bridge in the kit can have the formula

B-L-T,

wherein B represents the small molecule ligand, L represents the linker, and T represents the targeting moiety, and wherein L comprises a structure having the formula

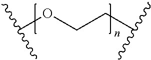

wherein n is an integer from 0 to 200. In other embodiments, n can be an integer from 0 to 150, an integer from 0 to 110, an integer from 0 to 20, an integer from 15 to 20, an integer from 15 to 110, or any other value or range of integers described herein for n.

A “pharmaceutically acceptable salt” of a small molecule ligand linked to a targeting moiety by a linker is contemplated. As used herein, the term “pharmaceutically acceptable salt” refers to those salts whose counter ions may be used in pharmaceuticals. Such salts include 1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or 2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like. Pharmaceutically acceptable salts are well-known to those skilled in the art, and any such pharmaceutically acceptable salt may be contemplated in connection with the embodiments described herein.

In various embodiments, suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

In various embodiments, suitable base salts are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

In one illustrative aspect, the compound, or a pharmaceutically salt thereof, the first conjugate, or a pharmaceutically acceptable salt thereof, or the second conjugate, or a pharmaceutically acceptable salt thereof, described herein may contain one or more chiral centers, or may otherwise be capable of existing as multiple stereoisomers. Accordingly, various embodiments may include pure stereoisomers as well as mixtures of stereoisomers, such as enantiomers, diastereomers, and enantiomerically or diastereomerically enriched mixtures. In one aspect, the compound, or pharmaceutically acceptable salt thereof, the first conjugate, or pharmaceutically acceptable salt thereof, the second conjugate, or pharmaceutically acceptable salt thereof, described herein may be capable of existing as geometric isomers. Accordingly, various embodiments may include pure geometric isomers or mixtures of geometric isomers.

In some aspects, the compound, or pharmaceutically acceptable salt thereof, the first conjugate, or pharmaceutically acceptable salt thereof, the second conjugate, or pharmaceutically acceptable salt thereof, described herein may exist in unsolvated forms as well as solvated forms, including hydrated forms. In general, the solvated forms are equivalent to unsolvated forms and are encompassed within the scope of the present invention.

The methods described herein also utilize cytotoxic T lymphocytes engineered to express a chimeric antigen receptor (CAR) that recognizes and binds to the targeting moiety (e.g., FITC, DNP, or TNP) of the bridge. In one embodiment, the CARs described herein comprise three domains including 1) a recognition region (e.g., a single chain fragment variable (scFv) region of an antibody) which recognizes and binds to the targeting moiety with specificity, 2) a co-stimulation domain which enhances the proliferation and survival of the T lymphocytes, and 3) an activation signaling domain which generates a cytotoxic T lymphocyte activation signal.

In various aspects, as non-limiting examples, scFv regions of antibodies that bind a folate, DUPA, a CAIX ligand, an NK-1R ligand, a ligand of gamma glutamyl transpeptidase, or a CCK2R ligand can be used. In illustrative embodiments, the scFv regions can be prepared from (i) an antibody known in the art that binds a targeting moiety, (ii) an antibody newly prepared using a selected targeting moiety, such as a hapten, and (iii) sequence variants derived from the scFv regions of such antibodies, e.g., scFv regions having at least about 80%, at least about 90%, at least about 95%, at least about 97%, at least about 98%, at least about 99%, or at least about 99.5% sequence identity to the amino acid sequence of the scFv region from which they are derived.

In any embodiments described herein, the binding portion of the CAR can be, for example, a single chain fragment variable region (scFv) of an antibody, an Fab, Fv, Fc, or (Fab′)2 fragment, and the like.

In one aspect, the co-stimulation domain serves to enhance the proliferation and survival of the cytotoxic T lymphocytes upon binding of the CAR to a targeting moiety. Suitable co-stimulation domains include: 1) CD28, 2) CD137 (4-1BB), a member of the tumor necrosis factor (TNF) receptor family, 3) CD134 (OX40), a member of the TNFR-superfamily of receptors, and 4) CD278 (ICOS), a CD28-superfamily co-stimulatory molecule expressed on activated T cells, or combinations thereof. Suitable co-stimulation domains also include, but are not limited to, CD27, CD30, CD150, DAP10, and NKG2D, or combinations thereof. A skilled artisan will understand that sequence variants of these co-stimulation domains can be used without adversely impacting the invention, where the variants have the same or similar activity as the domain on which they are modeled. In various embodiments, such variants have at least about 80%, at least about 90%, at least about 95%, at least about 97%, at least about 98%, at least about 99%, or at least about 99.5% sequence identity to the amino acid sequence of the domain from which they are derived.

In an illustrative embodiment, the activation signaling domain serves to activate cytotoxic T lymphocytes upon binding of the CAR to a targeting moiety. Suitable activation signaling domains include the T cell CD3 chain and Fc receptor γ. The skilled artisan will understand that sequence variants of these noted activation signaling domains can be used where the variants have the same or similar activity as the domain on which they are modeled. In various embodiments, the variants have at least about 80%, at least about 90%, at least about 95%, at least about 97%, at least about 98%, at least about 99%, or at least about 99.5% sequence identity to the amino acid sequence of the domain from which they are derived.

In one aspect, constructs encoding the CARs are prepared using genetic engineering techniques. Such techniques are described in detail in Sambrook et al., “Molecular Cloning: A Laboratory Manual”, 3rd Edition, Cold Spring Harbor Laboratory Press, (2001), incorporated herein by reference. As an example, a plasmid or viral expression vector (e.g., a lentiviral vector, a retrovirus vector, sleeping beauty, and piggyback (transposon/transposase systems that include a non-viral mediated CAR gene delivery system)) can be prepared that encodes a fusion protein comprising a recognition region, one or more co-stimulation domains, and an activation signaling domain, in frame and linked in a 5′ to 3′ direction. In other embodiments, other arrangements are acceptable and include a recognition region, an activation signaling domain, and one or more co-stimulation domains. The placement of the recognition region in the fusion protein will generally be such that display of the region on the exterior of the cell is achieved. In one embodiment, the CARs may include additional elements, such as a signal peptide to ensure proper export of the fusion protein to the cell surface, a transmembrane domain to ensure the fusion protein is maintained as an integral membrane protein, and a hinge domain that imparts flexibility to the recognition region and allows strong binding to the targeting moiety.

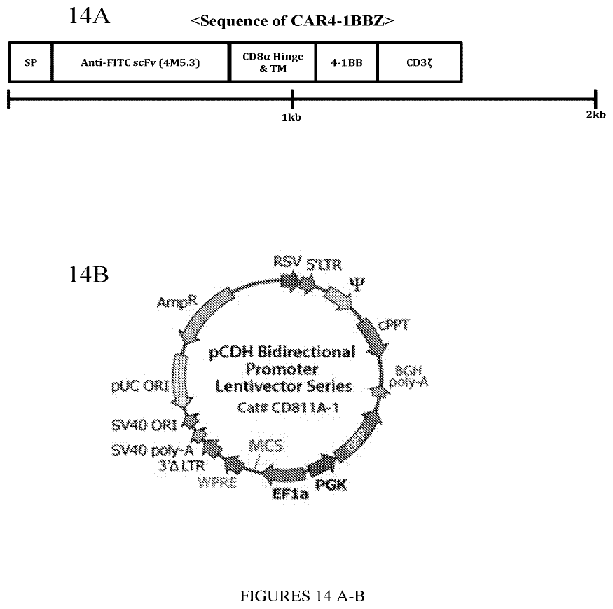

Diagrams of an exemplary CAR are shown in FIGS. 14A and B where the fusion protein sequence is incorporated into a lentivirus expression vector
and where “SP” is a signal peptide, the CAR is an anti-FITC CAR, a CD8a hinge and a transmembrane (TM) region is present, the co-stimulation domain is 4-1BB, and the activation signaling domain is CD3ζ. In one aspect, the nucleic acid sequence of the CAR insert is provided as SEQ ID NO:1 and the amino acid sequence of the insert is provided as SEQ ID NO:2.

In one embodiment, the CAR has a recognition region and the recognition region is a single chain fragment variable (scFv) region of an anti-FITC antibody, a co-stimulation domain and the co-stimulation domain is CD137 (4-1BB), and an activation signaling domain and the activation signaling domain is a T cell CD3 chain. It is well-known to the skilled artisan that an anti-FITC scFv and an anti-fluorescein scFv are equivalent terms.

In one embodiment, cytotoxic T lymphocytes can be genetically engineered to express CAR constructs by transfecting a population of the cytotoxic T lymphocytes with an expression vector encoding the CAR construct. Suitable methods for preparing a transduced population of T lymphocytes expressing a selected CAR construct are well-known to the skilled artisan, and are described in Sambrook et al., “Molecular Cloning: A Laboratory Manual”, 3rd Edition, Cold Spring Harbor Laboratory Press, (2001), incorporated herein by reference.

In various embodiments, CAR T cells comprising a nucleic acid of SEQ ID NO:1 or 3 are provided. In another embodiment, CAR T cells comprising a polypeptide of SEQ ID NO:2 are provided. In another illustrative aspect, an isolated nucleic acid comprising SEQ ID NO:1 or 3 and encoding a chimeric antigen receptor is provided. In yet another embodiment, a chimeric antigen receptor polypeptide comprising SEQ ID NO:2 is provided. In another embodiment, a vector is provided comprising SEQ ID NO:1 or 3. In another aspect, a lentiviral vector is provided comprising SEQ ID NO:1 or 3. In another embodiment, SEQ ID NO:2 can comprise or consist of human or humanized amino acid sequences.

In each of these embodiments, variant nucleic acid sequences or amino acid sequences having at least about 80%, at least about 90%, at least about 95%, at least about 97%, at least about 98%, at least about 99%, or at least about 99.5% sequence identity to SEQ ID NO: 1, SEQ ID NO:2, or SEQ ID NO:3 are contemplated. In another embodiment, the nucleic acid sequence can be a variant nucleic acid sequence having at least about 80%, at least about 90%, at least about 95%, at least about 97%, at least about 98%, at least about 99%, or at least about 99.5% sequence identity to SEQ ID NO:1 or 3 as long as the variant sequence encodes a polypeptide of SEQ ID NO:2. In another embodiment, the nucleic acid or amino acid sequence can be a variant nucleic acid or amino acid sequence having at least about 80%, at least about 90%, at least about 95%, at least about 97%, at least about 98%, at least about 99%, or at least about 99.5% sequence identity to SEQ ID NO:1 or SEQ ID NO:2 or SEQ ID NO:3 along a stretch of 200 nucleic acids or 200 amino acids of SEQ ID NO:1 or SEQ ID NO:2 or SEQ ID NO:3. Determination of percent identity or similarity between sequences can be done, for example, by using the GAP program (Genetics Computer Group, software; now available via Accelrys on http://www.accelrys.com), and alignments can be done using, for example, the ClustalW algorithm (VNTI software, InforMax Inc.). A sequence database can be searched using the nucleic acid or amino acid sequence of interest. Algorithms for database searching are typically based on the BLAST software (Altschul et al., 1990). In some embodiments, the percent identity can be determined along the full-length of the nucleic acid or amino acid sequence.

Also within the scope of the invention are nucleic acids complementary to the nucleic acid represented by SEQ ID NO:1 or 3, and those that hybridize to the nucleic acid represented by SEQ ID NO:1 or 3 or those that hybridize to its complement under highly stringent conditions. In accordance with the invention “highly stringent conditions” means hybridization at 65° C. in 5×SSPE and 50% formamide, and washing at 65° C. in 0.5×SSPE. Conditions for high stringency, low stringency and moderately stringent hybridization are described in Sambrook et al., “Molecular Cloning: A Laboratory Manual”, 3rd Edition, Cold Spring Harbor Laboratory Press, (2001), incorporated herein by reference. In some illustrative aspects, hybridization occurs along the full-length of the nucleic acid.

In one embodiment, the cytotoxic T lymphocytes used to prepare the CAR T cells, used in the methods described herein, can be autologous cells, although heterologous cells can also be used, such as when the patient being treated has received high-dose chemotherapy or radiation treatment to destroy the patient's immune system. In one embodiment, allogenic cells can be used.

In one aspect, the cytotoxic lymphocytes T can be obtained from a patient by means well-known in the art. For example, cytotoxic T cells can be obtained by collecting peripheral blood from the patient, subjecting the blood to Ficoll density gradient centrifugation, and then using a negative T cell isolation kit (such as EasySep™ T Cell Isolation Kit) to isolate a population of cytotoxic T cells from the peripheral blood. In one illustrative embodiment, the population of cytotoxic T lymphocytes need not be pure and may contain other cells such as other T cells, monocytes, macrophages, natural killer cells, and B cells. In one aspect, the population being collected can comprise at least about 90% of the selected cell type, at least about 91%, 92%, 93%, 94%, 95%, 96%, 97%, 98%, 99%, or 100% of the selected cell type.

In one embodiment, after the cytotoxic T lymphocytes are obtained, the cells are cultured under conditions that promote the activation of the cells. In this embodiment, the culture conditions may be such that the cells can be administered to a patient without concern for reactivity against components of the culture medium. For example, the culture conditions may not include bovine serum products, such as bovine serum albumin. In one illustrative aspect, the activation can be achieved by introducing known activators into the culture medium, such as anti-CD3 antibodies in the case of cytotoxic T cells. Other suitable activators include anti-CD28 antibodies. In one aspect, the population of lymphocytes can be cultured under conditions promoting activation for about 1 to about 4 days. In one embodiment, the appropriate level of activation can be determined by cell size, proliferation rate, or activation markers determined by flow cytometry.

In one illustrative embodiment, after the population of cytotoxic T lymphocytes has been cultured under conditions promoting activation, the cells can be transfected with an expression vector encoding a CAR. Suitable vectors and transfection methods are described above. In one aspect, after transfection, the cells can be immediately administered to the patient or the cells can be cultured for at least about 1, 2, 3, 4, 5, 6, 7, 8, 9, 10, 11, 12, 13, 14, 15, 16, 17, 18 or more days, or between about 5 and about 12 days, between about 6 and about 13 days, between about 7 and about 14 days, or between about 8 and about 15 days, for example, to allow time for the cells to recover from the transfection. Suitable culture conditions can be similar to the conditions under which the cells were cultured for activation either with or without the agent that was used to promote activation.

Thus, as described above, in one illustrative aspect, the methods of treatment described herein can further comprise 1) obtaining a population of autologous or heterologous cytotoxic T lymphocytes, 2) culturing the T lymphocytes under conditions that promote the activation of the cells, and 3) transfecting the lymphocytes with an expression vector encoding a CAR to form CAR T cells.

In one illustrative embodiment, when the cells have been transfected and activated a composition comprising the CAR T cells can be prepared and administered to the patient. In one embodiment, culture media that lacks any animal products, such as bovine serum, can be used. In another embodiment, tissue culture conditions typically used by the skilled artisan to avoid contamination with bacteria, fungi and mycoplasma can be used. In an exemplary embodiment, prior to being administered to a patient, the cells are pelleted, washed, and resuspended in a pharmaceutically acceptable carrier or diluent. Exemplary compositions comprising CAR-expressing cytotoxic T lymphocytes include compositions comprising the cells in sterile 290 mOsm saline, in infusible cryomedia (containing Plasma-Lyte A, dextrose, sodium chloride injection, human serum albumin and DMSO), in 0.9% NaCl with 2% human serum albumin, or in any other sterile 290 mOsm infusible materials. Alternatively, depending on the identity of the culture medium, the CAR T cells can be administered in the culture media as the composition, or concentrated and resuspended in the culture medium before administration. The CAR T cell composition can be administered to the patient via any suitable means, such as parenteral administration, e.g., intradermally, subcutaneously, intramuscularly, intraperitoneally, intravenously, or intrathecally.

In one aspect, the total number of CAR T cells and the concentration of the cells in the composition administered to the patient will vary depending on a number of factors including the type of cytotoxic T lymphocytes being used, the binding specificity of the CAR, the identity of the targeting moiety and the small molecule ligand, the identity of the cancer, the location of the cancer in the patient, the means used to administer the compositions to the patient, and the health, age and weight of the patient being treated. However, suitable compositions comprising transduced CAR T cells include those having a volume of between about 5 ml and about 200 ml, containing from about 1×10⁵ to about 1×10¹⁵ transduced CAR T cells. Typical compositions comprise a volume of between about 10 ml and about 125 ml, containing from about 1×10⁷ to about 1×10¹⁰ CAR T cells. An exemplary composition comprises about 1×10⁹ CART cells in a volume of about 100 ml. In one aspect, a single dose or multiple doses of the CAR T cells can be administered to the patient.

In various embodiments, the cancer to be treated is a carcinoma, a sarcoma, a lymphoma, a melanoma, a mesothelioma, a nasopharyngeal carcinoma, a leukemia, an adenocarcinoma, or a myeloma. In other embodiments, the cancer may be lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, prostate cancer, chronic leukemia, acute leukemia, a lymphocytic lymphoma, pleural mesothelioma, cancer of the bladder, Burkitt's lymphoma, cancer of the ureter, cancer of the kidney, renal cell carcinoma, carcinoma of the renal pelvis, a neoplasm of the central nervous system (CNS), primary CNS lymphoma, a spinal axis tumor, a brain stem glioma, a pituitary adenoma, or an adenocarcinoma of the gastroesophageal junction.

In some aspects of these embodiments, the cancer is a folate receptor expressing cancer. In some aspects of these embodiments, the cancer is an endometrial cancer, a non-small cell lung cancer, an ovarian cancer, or a triple-negative breast cancer. In another embodiment, the cancer being imaged is a tumor. In another embodiment, the cancer is malignant.

The compound, or pharmaceutically acceptable salt thereof, the first conjugate, or pharmaceutically acceptable salt thereof, the second conjugate, or pharmaceutically acceptable salt thereof, or CAR T cell composition described herein can be administered to the patient using any suitable method known in the art. As described herein, the term “administering” or “administered” includes all means of introducing the compound, or pharmaceutically acceptable salt thereof, the first conjugate, or pharmaceutically acceptable salt thereof, the second conjugate, or pharmaceutically acceptable salt thereof, or CAR T cell composition to the patient, including, but not limited to, oral (po), intravenous (iv), intramuscular (im), subcutaneous (sc), transdermal, and the like. The compound, or pharmaceutically acceptable salt thereof, the first conjugate, or pharmaceutically acceptable salt thereof, the second conjugate, or pharmaceutically acceptable salt thereof, described herein may be administered in unit dosage forms and/or formulations containing conventional nontoxic pharmaceutically-acceptable carriers, adjuvants, and vehicles.

In one aspect, the compound, or pharmaceutically acceptable salt thereof, the first conjugate, or pharmaceutically acceptable salt thereof, the second conjugate, or pharmaceutically acceptable salt thereof, or CAR T cell composition as described herein may be administered directly into the blood stream, into muscle, or into an internal organ. Suitable routes for such parenteral administration include intravenous, intraarterial, intraperitoneal, intrathecal, epidural, intracerebroventricular, intraurethral, intrastenal, intracranial, intratumoral, intramuscular and subcutaneous delivery. In one embodiment, means for parenteral administration include needle (including microneedle) injectors, needle-free injectors and infusion techniques.

In one illustrative aspect, parenteral formulations are typically aqueous solutions which may contain carriers or excipients such as salts, carbohydrates and buffering agents (preferably at a pH of from 3 to 9), but they may be more suitably formulated as a sterile non-aqueous solution or as a dried form to be used in conjunction with a suitable vehicle such as sterile, pyrogen-free water or sterile saline. In other embodiments, any of the liquid formulations described herein may be adapted for parenteral administration as described herein. The preparation under sterile conditions, by lyophilization to produce a sterile lyophilized powder for a parenteral formulation, may readily be accomplished using standard pharmaceutical techniques well-known to those skilled in the art. In one embodiment, the solubility of the compound, or pharmaceutically acceptable salt thereof, the first conjugate, or pharmaceutically acceptable salt thereof, the second conjugate, or pharmaceutically acceptable salt thereof, used in the preparation of a parenteral formulation may be increased by the use of appropriate formulation techniques, such as the incorporation of solubility-enhancing agents.

In some embodiments, the rate of tumor lysis can be regulated by adjusting the concentration of the first conjugate, or pharmaceutically acceptable salt thereof, the second conjugate, or pharmaceutically acceptable salt thereof, or both the first and second conjugate, or pharmaceutically acceptable salt thereof. Accordingly, by varying the concentration of the first conjugate, or pharmaceutically acceptable salt thereof, the second conjugate, or pharmaceutically acceptable salt thereof, or both the first and second conjugate, or pharmaceutically acceptable salt thereof, the cytotoxicity of the CAR T cell composition can be regulated. In some embodiments, the cytotoxicity of the CAR T cell composition can be balanced against the risk of tumor lysis syndrome, or cytokine release syndrome (CRS), as described herein by adjusting the concentration of the first conjugate, or pharmaceutically acceptable salt thereof, the second conjugate, or pharmaceutically acceptable salt thereof, or both the first and second conjugate, or pharmaceutically acceptable salt thereof. It will be appreciated that the concentration of the first conjugate, or pharmaceutically acceptable salt thereof, the second conjugate, or pharmaceutically acceptable salt thereof, or both the first and second conjugate, or pharmaceutically acceptable salt thereof can be a function of the amount or dose of the first conjugate, or pharmaceutically acceptable salt thereof, the second conjugate, or pharmaceutically acceptable salt thereof, or both the first and second conjugate, or pharmaceutically acceptable salt thereof that is administered to the patient.

The amount of the compound, or pharmaceutically acceptable salt thereof, the first conjugate, or pharmaceutically acceptable salt thereof, the second conjugate, or pharmaceutically acceptable salt thereof, to be administered to the patient can vary significantly depending on the cancer being treated, the route of administration of the compound, or pharmaceutically acceptable salt thereof, the first conjugate, or pharmaceutically acceptable salt thereof, the second conjugate, or pharmaceutically acceptable salt thereof, and the tissue distribution. The amount to be administered to a patient can be based on body surface area, mass, and physician assessment. In various embodiments, amounts to be administered can range, for example, from about 0.05 mg to about 30 mg, 0.05 mg to about 25.0 mg, about 0.05 mg to about 20.0 mg, about 0.05 mg to about 15.0 mg, about 0.05 mg to about 10.0 mg, about 0.05 mg to about 9.0 mg, about 0.05 mg to about 8.0 mg, about 0.05 mg to about 7.0 mg, about 0.05 mg to about 6.0 mg, about 0.05 mg to about 5.0 mg, about 0.05 mg to about 4.0 mg, about 0.05 mg to about 3.0 mg, about 0.05 mg to about 2.0 mg, about 0.05 mg to about 1.0 mg, about 0.05 mg to about 0.5 mg, about 0.05 mg to about 0.4 mg, about 0.05 mg to about 0.3 mg, about 0.05 mg to about 0.2 mg, about 0.05 mg to about 0.1 mg, about 0.01 mg to about 2 mg, about 0.3 mg to about 10 mg, about 0.1 mg to about 20 mg, or about 0.8 to about 3 mg. One of skill in the art will readily appreciate that the dose may vary within the various ranges provided above based on the factors noted above, and may be at the physician's discretion.

In other embodiments, the dose of the compound, or pharmaceutically acceptable salt thereof, the first conjugate, or pharmaceutically acceptable salt thereof, the second conjugate, or pharmaceutically acceptable salt thereof, can range, for example, from about 50 nmol/kg to about 3000 nmol/kg of patient body weight, about 50 nmol/kg to about 2000 nmol/kg, about 50 nmol/kg to about 1000 nmol/kg, about 50 nmol/kg to about 900 nmol/kg, about 50 nmol/kg to about 800 nmol/kg, about 50 nmol/kg to about 700 nmol/kg, about 50 nmol/kg to about 600 nmol/kg, about 50 nmol/kg to about 500 nmol/kg, about 50 nmol/kg to about 400 nmol/kg, about 50 nmol/kg to about 300 nmol/kg, about 50 nmol/kg to about 200 nmol/kg, about 50 nmol/kg to about 100 nmol/kg, about 100 nmol/kg to about 300 nmol/kg, about 100 nmol/kg to about 500 nmol/kg, about 100 nmol/kg to about 1000 nmol/kg, about 100 nmol/kg to about 2000 nmol/kg of patient body weight. In other embodiments, the dose may be about 100 nmol/kg, about 150 nmol/kg, about 200 nmol/kg, about 250 nmol/kg, about 300 nmol/kg, about 350 nmol/kg, about 400 nmol/kg, about 450 nmol/kg, about 500 nmol/kg, about 600 nmol/kg, about 700 nmol/kg, about 800 nmol/kg, about 900 nmol/kg, about 1000 nmol/kg, about 2000 nmol/kg, or about 3000 nmol/kg of patient body weight. In these embodiments, “kg” is kilograms of patient body weight. In one aspect, a single dose or multiple doses of the compound, or pharmaceutically acceptable salt thereof, the first conjugate, or pharmaceutically acceptable salt thereof, the second conjugate, or pharmaceutically acceptable salt thereof, may be administered to the patient.

In another embodiment, between about 20 ug/kg of patient body weight and about 3 mg/kg of patient body weight of the compound, or pharmaceutically acceptable salt thereof, the first conjugate, or pharmaceutically acceptable salt thereof, the second conjugate, or pharmaceutically acceptable salt thereof, is administered to the patient. In another aspect, amounts can be between about 0.2 mg/kg of patient body weight and about 0.4 mg/kg of patient body weight, or can be about 50 ug/kg of patient body weight. In one aspect, a single dose or multiple doses of the compound, or pharmaceutically acceptable salt thereof, the first conjugate, or pharmaceutically acceptable salt thereof, the second conjugate, or pharmaceutically acceptable salt thereof, may be administered to the patient.

In one embodiment, the small molecule ligand linked to the targeting moiety can be administered to the patient before the CAR T cell composition. In another embodiment, the small molecule ligand linked to the targeting moiety can be administered to the patient at the same time as the CAR T cell composition, but in different formulations. In yet another embodiment, the small molecule ligand linked to the targeting moiety can be administered to the patient after the CAR T cell composition.

In one illustrative aspect, the timing between the administration of CAR T cells and the small molecule linked to the targeting moiety may vary widely depending on factors that include the type of CAR T cells being used, the binding specificity of the CAR, the identity of the targeting moiety and the ligand, the identity of the cancer, the location in the patient of the cancer, the means used to administer to the patient the CAR T cells and the small molecule ligand linked to the targeting moiety, and the health, age, and weight of the patient. In one aspect, the small molecule ligand linked to the targeting moiety can be administered before or after the CAR T cells, such as within about 3, 6, 9, 12, 15, 18, 21, or 24 hours, or within about 0.5, 1, 1.5, 2, 2.5, 3, 4 5, 6, 7, 8, 9, 10 or more days.

In some embodiments, the rate of tumor lysis can be regulated by adjusting the rate of administration of the first conjugate, or pharmaceutically acceptable salt thereof, the second conjugate, or pharmaceutically acceptable salt thereof, or both the first and second conjugate, or pharmaceutically acceptable salt thereof. Accordingly, by varying the rate of administration of the first conjugate, or pharmaceutically acceptable salt thereof, the second conjugate, or pharmaceutically acceptable salt thereof, or both the first and second conjugate, or pharmaceutically acceptable salt thereof, the cytotoxicity of the CAR T cell composition can be regulated. In some embodiments, the cytotoxicity of the CAR T cell composition can be balanced against the risk of tumor lysis syndrome, or cytokine release syndrome (CRS), as described herein by adjusting the rate of administration of the first conjugate, or pharmaceutically acceptable salt thereof, the second conjugate, or pharmaceutically acceptable salt thereof, or both the first and second conjugate, or pharmaceutically acceptable salt thereof. It will be appreciated that the rate of administration of the first conjugate, or pharmaceutically acceptable salt thereof, the second conjugate, or pharmaceutically acceptable salt thereof, or both the first and second conjugate, or pharmaceutically acceptable salt thereof can be a function of any applicable dosing schedule known in the art. For example, the rate of administration can be a function of a dosing schedule that is based on continuous dosing, once per day dosing (a.k.a qd), twice per day dosing (a.k.a. bid), three times per day dosing (a.k.a. tid), twice per week (a.k.a. BIW), three times per week (a.k.a. TIW), once weekly, and the like. It will be appreciated that the dosing schedule selected for first conjugate, or pharmaceutically acceptable salt thereof, the second conjugate, or pharmaceutically acceptable salt thereof, or both the first and second conjugate, or pharmaceutically acceptable salt thereof can be applied in connection with the concentration to regulate the cytotoxicity of the CAR T cell composition. In some embodiments, the cytotoxicity of the CAR T cell composition can be balanced against the risk of tumor lysis syndrome, or cytokine release syndrome (CRS), as described herein by adjusting the dosing schedule in connection with the concentration of the first conjugate, or pharmaceutically acceptable salt thereof, the second conjugate, or pharmaceutically acceptable salt thereof, or both the first and second conjugate, or pharmaceutically acceptable salt thereof.

In one embodiment of the methods described herein, the cancer is imaged prior to administration to the patient of the compound, or pharmaceutically acceptable salt thereof, the first conjugate, or pharmaceutically acceptable salt thereof, the second conjugate, or pharmaceutically acceptable salt thereof, or prior to administration of the CAR T cell composition to the patient. In one illustrative embodiment, imaging occurs by PET imaging. In other illustrative embodiments imaging occurs by MRI imaging or SPECT/CT imaging. It is appreciated by one skilled in the art that the imaging method can be any suitable imaging method known in the art.

In any of the embodiments described herein, cytokine release resulting in ‘off-target’ toxicity in the patient may not occur even though CAR T cell toxicity to the cancer occurs. In any embodiment described herein, ‘off-target’ tissue toxicity may not occur in the patient even though CAR T cell toxicity to the cancer occurs. In any embodiment described herein, the cancer may comprise a tumor, and tumor size may be reduced in the patient, even though ‘off-target’ toxicity does not occur.

### EXAMPLES

### Example 1

### Generation of Lentiviral Vector Encoding CAR Gene

An overlap PCR method was used to generate CAR constructs comprising scFv against fluorescein. scFV against fluorescein, 4M5.3 (Kd=270 fM, 762 bp) derived from anti-fluorescein (4-4-20) antibody was synthesized. Sequence encoding the human CD8α signal peptide (SP, 63 bp), the hinge, and transmembrane region (249 bp), the cytoplasmic domain of 4-1BB (CD137, 141 bp) and the CD3ζ chain (336 bp), as shown in FIG. 14A, were fused with the anti-fluorescein scFV by overlapping PCR. The resulting CAR construct (1551 bp) was inserted into EcoRI/NotI cleaved lentiviral expression vector pCDH-EF1-MCS-(PGK-GFP) (FIG. 14B, System Biosciences). The sequence of the CAR construct in lentiviral vector was confirmed by DNA sequencing.

An exemplary CAR nucleic acid coding sequence may comprise:

(SEQ ID NO: 1)
ATGGCCTTACCAGTGACCGCCTTGCTCCTGCCGCTGGCCTTGCTGCTCC
ACGCCGCCAGGCCGGATGTCGTGATGACCCAGACCCCCCTCAGCCTCCC
AGTGTCCCTCGGTGACCAGGCTTCTATTAGTTGCAGATCCAGCCAGTCC
CTCGTGCACTCTAACGGTAATACCTACCTGAGATGGTATCTCCAGAAGC
CCGGACAGAGCCCTAAGGTGCTGATCTACAAAGTCTCCAACCGGGTGTC
TGGAGTCCCTGACCGCTTCTCAGGGAGCGGTTCCGGCACCGACTTCACC
CTGAAGATCAACCGGGTGGAGGCCGAAGACCTCGGCGTCTATTTCTGCT
CTCAGAGTACACATGTGCCCTGGACCTTCGGCGGAGGGACCAAGCTGGA
GATCAAAAGCTCCGCAGACGATGCCAAGAAAGATGCCGCTAAGAAAGAC
GATGCTAAGAAAGACGATGCAAAGAAAGACGGTGGCGTGAAGCTGGATG
AAACCGGAGGAGGTCTCGTCCAGCCAGGAGGAGCCATGAAGCTGAGTTG
CGTGACCAGCGGATTCACCTTTGGGCACTACTGGATGAACTGGGTGCGA
CAGTCCCCAGAGAAGGGGCTCGAATGGGTCGCTCAGTTCAGGAACAAAC
CCTACAATTATGAGACATACTATTCAGACAGCGTGAAGGGCAGGTTTAC
TATCAGTAGAGACGATTCCAAATCTAGCGTGTACCTGCAGATGAACAAT
CTCAGGGTCGAAGATACAGGCATCTACTATTGCACAGGGGCATCCTATG
GTATGGAGTATCTCGGTCAGGGGACAAGCGTCACAGTCAGTTTCGTGCC
GGTCTTCCTGCCAGCGAAGCCCACCACGACGCCAGCGCCGCGACCACCA
ACACCGGCGCCCACCATCGCGTCGCAGCCCCTGTCCCTGCGCCCAGAGG
CGTGCCGGCCAGCGGCGGGGGGCGCAGTGCACACGAGGGGGCTGGACTT
CGCCTGTGATATCTACATCTGGGCGCCCTTGGCCGGGACTTGTGGGGTC
CTTCTCCTGTCACTGGTTATCACCCTTTACTGCAACCACAGGAACCGTT
TCTCTGTTGTTAAACGGGGCAGAAAGAAACTCCTGTATATATTCAAACA
ACCATTTATGAGACCAGTACAAACTACTCAAGAGGAAGATGGCTGTAGC
TGCCGATTTCCAGAAGAAGAAGAAGGAGGATGTGAACTGAGAGTGAAGT
TCAGCAGGAGCGCAGACGCCCCCGCGTACCAGCAGGGCCAGAACCAGCT
CTATAACGAGCTCAATCTAGGACGAAGAGAGGAGTACGATGTTTTGGAC
AAGAGACGTGGCCGGGACCCTGAGATGGGGGGAAAGCCGAGAAGGAAGA
ACCCTCAGGAAGGCCTGTACAATGAACTGCAGAAAGATAAGATGGCGGA
GGCCTACAGTGAGATTGGGATGAAAGGCGAGCGCCGGAGGGGCAAGGGG
CACGATGGCCTTTACCAGGGTCTCAGTACAGCCACCAAGGACACCTACG
ACGCCCTTCACATGCAGGCCCTGCCCCCTCGCTAA.

In the exemplary nucleic acid sequence shown above, the first ATG is the start codon. An exemplary CAR amino acid sequence may comprise:

(SEQ ID NO: 2)
MALPVTALLLPLALLLHAARPDVVMTQTPLSLPVSLGDQASISCRSSQS
LVHSNGNTYLRWYLQKPGQSPKVLIYKVSNRVSGVPDRFSGSGSGTDFT
LKINRVEAEDLGVYFCSQSTHVPWTFGGGTKLEIKSSADDAKKDAAKKD
DAKKDDAKKDGGVKLDETGGGLVQPGGAMKLSCVTSGFTFGHYWMNWVR
QSPEKGLEWVAQFRNKPYNYETYYSDSVKGRFTISRDDSKSSVYLQMNN
LRVEDTGIYYCTGASYGMEYLGQGTSVTVSFVPVFLPAKPTTTPAPRPP
TPAPTIASQPLSLRPEACRPAAGGAVHTRGLDFACDIYIWAPLAGTCGV
LLLSLVITLYCNHRNRFSVVKRGRKKLLYIFKQPFMRPVQTTQEEDGCS
CRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKG
HDGLYQGLSTATKDTYDALHMQALPPR

An exemplary insert may comprise:

(SEQ ID NO: 3)
GCCACCATGGCCTTACCAGTGACCGCCTTGCTCCTGCCGCTGGCCTTGC
TGCTCCACGCCGCCAGGCCGGATGTCGTGATGACCCAGACCCCCCTCAG
CCTCCCAGTGTCCCTCGGTGACCAGGCTTCTATTAGTTGCAGATCCAGC
CAGTCCCTCGTGCACTCTAACGGTAATACCTACCTGAGATGGTATCTCC
AGAAGCCCGGACAGAGCCCTAAGGTGCTGATCTACAAAGTCTCCAACCG
GGTGTCTGGAGTCCCTGACCGCTTCTCAGGGAGCGGTTCCGGCACCGAC
TTCACCCTGAAGATCAACCGGGTGGAGGCCGAAGACCTCGGCGTCTATT
TCTGCTCTCAGAGTACACATGTGCCCTGGACCTTCGGCGGAGGGACCAA
GCTGGAGATCAAAAGCTCCGCAGACGATGCCAAGAAAGATGCCGCTAAG
AAAGACGATGCTAAGAAAGACGATGCAAAGAAAGACGGTGGCGTGAAGC
TGGATGAAACCGGAGGAGGTCTCGTCCAGCCAGGAGGAGCCATGAAGCT
GAGTTGCGTGACCAGCGGATTCACCTTTGGGCACTACTGGATGAACTGG
GTGCGACAGTCCCCAGAGAAGGGGCTCGAATGGGTCGCTCAGTTCAGGA
ACAAACCCTACAATTATGAGACATACTATTCAGACAGCGTGAAGGGCAG
GTTTACTATCAGTAGAGACGATTCCAAATCTAGCGTGTACCTGCAGATG
AACAATCTCAGGGTCGAAGATACAGGCATCTACTATTGCACAGGGGCAT
CCTATGGTATGGAGTATCTCGGTCAGGGGACAAGCGTCACAGTCAGTTT
CGTGCCGGTCTTCCTGCCAGCGAAGCCCACCACGACGCCAGCGCCGCGA
CCACCAACACCGGCGCCCACCATCGCGTCGCAGCCCCTGTCCCTGCGCC
CAGAGGCGTGCCGGCCAGCGGCGGGGGGCGCAGTGCACACGAGGGGGCT
GGACTTCGCCTGTGATATCTACATCTGGGCGCCCTTGGCCGGGACTTGT
GGGGTCCTTCTCCTGTCACTGGTTATCACCCTTTACTGCAACCACAGGA
ACCGTTTCTCTGTTGTTAAACGGGGCAGAAAGAAACTCCTGTATATATT
CAAACAACCATTTATGAGACCAGTACAAACTACTCAAGAGGAAGATGGC
TGTAGCTGCCGATTTCCAGAAGAAGAAGAAGGAGGATGTGAACTGAGAG
TGAAGTTCAGCAGGAGCGCAGACGCCCCCGCGTACCAGCAGGGCCAGAA
CCAGCTCTATAACGAGCTCAATCTAGGACGAAGAGAGGAGTACGATGTT
TTGGACAAGAGACGTGGCCGGGACCCTGAGATGGGGGGAAAGCCGAGAA
GGAAGAACCCTCAGGAAGGCCTGTACAATGAACTGCAGAAAGATAAGAT
GGCGGAGGCCTACAGTGAGATTGGGATGAAAGGCGAGCGCCGGAGGGGC
AAGGGGCACGATGGCCTTTACCAGGGTCTCAGTACAGCCACCAAGGACA
CCTACGACGCCCTTCACATGCAGGCCCTGCCCCCTCGCTAA

In the exemplary insert shown above, the first GCCACC sequence may be a restriction enzyme cleavage site, followed by the ATG start codon. An exemplary insert amino acid sequence may comprise:

(SEQ ID NO: 4)
ATMALPVTALLLPLALLLHAARPDVVMTQTPLSLPVSLGDQASISCRSS
QSLVHSNGNTYLRWYLQKPGQSPKVLIYKVSNRVSGVPDRFSGSGSGTD
FTLKINRVEAEDLGVYFCSQSTHVPWTFGGGTKLEIKSSADDAKKDAAK
KDDAKKDDAKKDGGVKLDETGGGLVQPGGAMKLSCVTSGFTFGHYWMNW
VRQSPEKGLEWVAQFRNKPYNYETYYSDSVKGRFTISRDDSKSSVYLQM
NNLRVEDTGIYYCTGASYGMEYLGQGTSVTVSFVPVFLPAKPTTTPAPR
PPTPAPTIASQPLSLRPEACRPAAGGAVHTRGLDFACDIYIWAPLAGTC
GVLLLSLVITLYCNHRNRFSVVKRGRKKLLYIFKQPFMRPVQTTQEEDG
CSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRG
KGHDGLYQGLSTATKDTYDALHMQALPPR

### Example 2

### Production of Lentivius Containing CAR Gene for Human T Cell Transduction

To prepare lentiviral virus containing an anti-fluorescein single chain fragment variable (scFv) CAR, a 293TN packaging cell line was co-transfected with the lentiviral vector encoding anti-fluorescein scFv CAR and a 2nd generation of a mixture of packaging plasmids (Cellecta). After 24 and 48 hours of transfection, supernatants containing the lentivirus with the CAR gene were harvested and virus particles were concentrated by the standard polyethylene glycol virus concentration method (Clontech) for future transduction with human T cells.

### Example 3

Isolation of Human T Cells from Human PBMC

T cells were isolated from human peripheral blood mononuclear cells (PBMC) by Ficoll density gradient centrifugation (GE Healthcare Lifesciences). After washing away remaining Ficoll solution, T cells were isolated by using an EasySep™ Human T Cell Isolation Kit (STEM CELL technologies). Purified T cells were cultured in TexMACS™ medium (Miltenyi Biotech Inc) with 1% penicillin and streptomycin sulfate in the presence of human IL-2 (100 IU/mL, Miltenyi Biotech Inc). T cells were cultured at density of 1×10⁶ cells/mL in multi-well plates. T cells were split and re-feed every 2-3 days.

### Example 4

### Transduction of Human T Cells

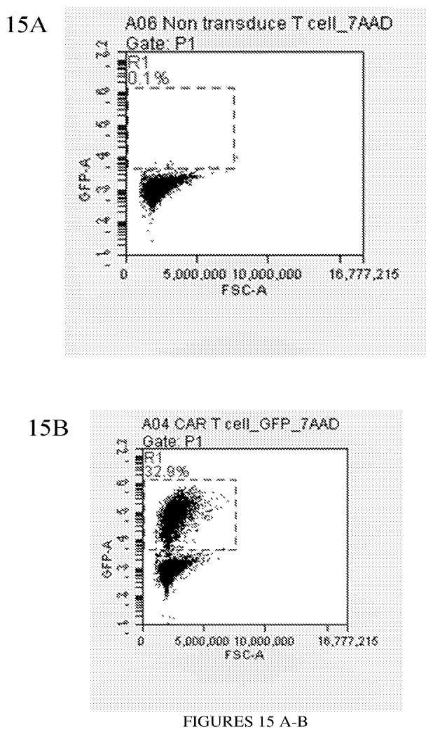

Isolated T cells were activated with Dynabeads coupled with anti-CD3/CD28 antibodies (Life Technologies) for 12-24 hours in the presence of human IL-2 (100 IU/mL), then transduced with lentivirus encoding an anti-fluorescein CAR gene. Cells were harvested after 72 hours and the expression of CAR on transduced T cells was identified by measuring GFP fluorescent cells using flow cytometry. As shown in FIG. 15A, non-transduced T cells do not show GFP expression. As shown in FIG. 15B, transduced T-cells express GFP.

### Example 5

### Synthesis of FITC-Folate

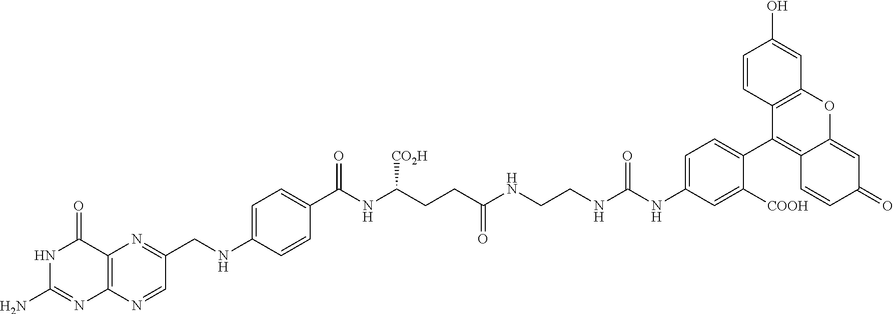 [SMILES: Nc1nc2ncc(CNc3ccc(C(=O)N[C@@H](CCC(=O)NCCNC(=O)Nc4ccc(-c5c6ccc(=O)cc-6oc6cc(O)ccc56)c(C(=O)O)c4)C(=O)O)cc3)nc2c(=O)[nH]1]

Folate-γ-ethylenediamine was coupled to fluorescein isothiocyanate (FITC) isomer I (Sigma-Aldrich) in anhydrous dimethylsulfoxide (DMF) in the presence of tetramethylguanidine and diisopropylamine. The crude product was loaded onto an Xterra RP18 preparative HPLC column (Waters) and eluted with gradient conditions starting with 99% 5 mM sodium phosphate (mobile phase A, pH 7.4) and 1% acetonitrile (mobile phase B) and reaching 90% A and 10% B in 10 min at a flow rate of 20 mL/min. Under these conditions, the FITC-folate main peak typically eluted at 27-50 min. The quality of the FITC-folate fraction was monitored by analytical reverse-phase HPLC with a UV detector. Fractions with greater than 98.0% purity (LCMS) were lyophilized to obtain the final FITC-folate product.

### Example 6

### Synthesis of FITC-PEG12-Folate

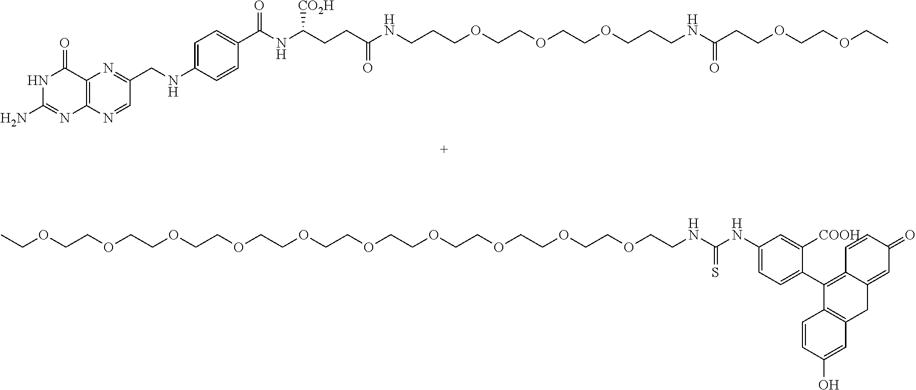

Universal polyethylene glycol (PEG) Nova Tag™ resin (0.2 g) was loaded into a peptide synthesis vessel and washed with isopropyl alcohol (i-PrOH) (3×10 mL) and dimethylformamide (DMF, 3×10 mL). 9-fluorenylmethoxycarbonyl (Fmoc) deprotection was carried out using 20% piperidine in DMF (3×10 mL). Kaiser tests were performed to assess reaction progress. To the vessel was then introduced a solution of Fmoc-L-glutamic acid 5-tert-butyl ester (Fmoc-Glu-(0-t-Bu)-OH) (23.5 mg) in DMF, N,N-diisopropylethylamine (i-Pr₂NEt) (4 equiv), and benzotriazol-1-yl-oxvtriyrrolidinonhosnhonium hexafluoronhosnhate (PyBOP) (2 equiv). Fmoc deprotection was carried out using 20% piperidine in DMF (3×10 mL). To the vessel was then introduced a solution of Ni¹⁰-TFA-Pte-OH (22.5 mg), DMF, i-Pr₂NEt (4 equiv), and PyBOP (2 equiv). Argon was bubbled for 2 h, and the resin was washed with DMF (3×3 mL) and i-PrOH (3×3 mL). After swelling the resin in dichloromethane (DCM), a solution of 1M hydroxybenzotriazole (HOBT) in DCM/trifluoroethane (TFE) (1:1) (2×3 mL) was added. Argon was bubbled for 1 h, the solvent was removed, and the resin was washed with DMF (3×3 mL) and i-PrOH (3×3 mL). After swelling the resin in DMF, a solution of Fmoc-NH-(PEG)₁₂-COOH (46.3 mg) in DMF, i-Pr₂NEt (4 equiv), and PyBOP (2 equiv) was added. Argon was bubbled for 2 h, and the resin was washed with DMF (3×3 mL) and i-PrOH (3×3 mL). Fmoc deprotection was carried out using 20% piperidine in DMF (3×10 mL). Kaiser tests were performed to assess reaction progress. To the vessel was then introduced a solution of FITC (Life Technologies 21.4 mg) in DMF and i-Pr₂NEt (4 equiv), then Argon was bubbled for 2 h, and the resin was washed with DMF (3×3 mL) and i-PrOH (3×3 mL). Then to the vessel was added 2% NH₂NH₂ in DMF (2×2 mL). The final compound was cleaved from the resin using a TFA:H₂O: triisopropylsilane (TIS) (95:2.5:2.5) (Cleavage Solution) and concentrated under vacuum. The concentrated product was precipitated in Et₂O and dried under vacuum. The crude product was purified using preparative RP-HPLC (mobile phase: A=10 mM ammonium acetate pH=7, B=ACN; method: 0% B to 30% B in 30 min at 13 mL/min). The pure fractions were pooled and freeze-dried, providing the FITC-PEG12-Folate.

### Example 7

### Synthesis of FITC-PEG20-Folate

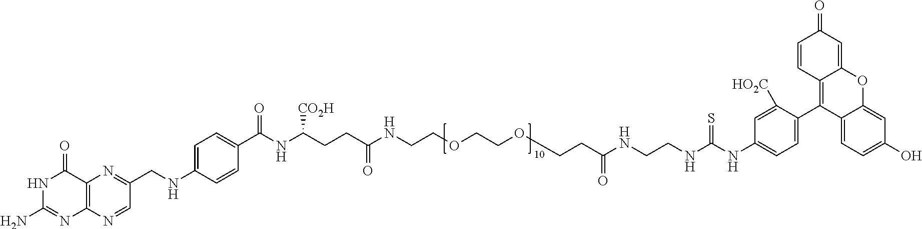

Ethylenediamine, polymer-bound (200-400 mesh)-resin (50 mg) was loaded into a peptide synthesis vessel and swollen with DCM (3 mL) followed by DMF (3 mL). To the vessel was then introduced the Fmoc-PEG₂₀-COOH solution (131 mg, 1.0 equiv) in DMF, i-Pr₂NEt (6.0 equiv), and PyBOP (4.0 equiv). Argon was bubbled for 6 h, the coupling solution was drained, and the resin was washed with DMF (3×10 mL) and i-PrOH (3×10 mL). Kaiser tests were performed to assess reaction progress. Fmoc deprotection was carried out using 20% piperidine in DMF (3×10 mL), before each amino acid coupling. The above sequence was repeated to complete the reaction with Fmoc-Glu-OtBu (72 mg, 2.0 equiv) and Tfa.Pteroic-acid (41 mg, 1.2 equiv) coupling steps. The resin was washed with 2% hydrazine in DMF 3×10 mL (5 min) to cleave the trifluoro-acetyl protecting group on pteroic acid and washed with i-PrOH (3×10 mL) followed by DMF (3×10 mL). The resin was dried under argon for 30 min. The folate-peptide was cleaved from the resin using the Cleavage Solution. 10 mL of the cleavage mixture was introduced and argon was bubbled for 1.5 h. The cleavage mixture was drained into a clean flask. The resin was washed 3 times with more cleavage mixture. The combined mixture was concentrated under reduced pressure to a smaller volume (˜5 mL) and precipitated in ethyl ether.

The precipitate was collected by centrifugation, washed with ethyl ether (3 times) and dried under high vacuum. The dried Folate-PEG₂₀-EDA (1.0 equiv) was treated with FITC (50 mg, 1.5 equiv) in DMSO and DIPEA at room temperature. Progress of the reaction monitored by LCMS. After 8 h the starting material was consumed to give the product. The crude reaction mixture was purified by preparative HPLC, (mobile phase A=10 mM Ammonium Acetate, pH=7; Organic phase B=Acetonitrile; Method: 0% B to 30% B in 35 minutes at 13 mL/min) and provided FITC-PEG20-Folate in 60% yield.

### Example 8

### Synthesis of FITC-PEG108-Folate

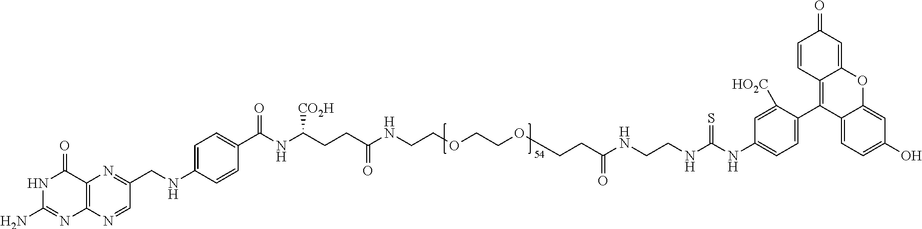

Ethylenediamine, polymer-bound (200-400 mesh)-resin (50 mg) was loaded in a peptide synthesis vessel and swollen with DCM (3 mL) followed by DMF (3 mL). To the vessel was then introduced the Fmoc-PEG₃₆-COOH solution (161 mg, 1.0 equiv) in DMF, i-Pr₂NEt (6.0 equiv), and PyBOP (4.0 equiv). Argon was bubbled for 6 h, the coupling solution was drained, and the resin was washed with DMF (3×10 mL) and i-PrOH (3×10 mL). Kaiser tests were performed to assess reaction progress. Fmoc deprotection was carried out using 20% piperidine in DMF (3×10 mL), before each amino acid coupling. The above sequence was repeated to complete reaction with 2× Fmoc-PEG₃₆-COOH (161 mg, 1.0 equiv), Fmoc-Glu-OtBu (72 mg, 2.0 equiv) and Tfa.Pteroic-acid (41.0 mg, 1.2 equiv) coupling steps. At the end the resin was washed with 2% hydrazine in DMF 3×10 mL (5 min) to cleave the trifluoro-acetyl protecting group on pteroic acid and washed with i-PrOH (3×10 mL) followed by DMF (3×10 mL). The resin was dried under argon for 30 min. Folate-peptide was cleaved from the resin using the Cleavage Solution. 10 mL of the cleavage mixture was introduced and argon was bubbled for 1.5 h. The cleavage mixture was drained into a clean flask. The resin was washed 3× with more Cleavage Solution. The combined mixture was concentrated under reduced pressure to a smaller volume (˜5 mL) and precipitated in ethyl ether.

The precipitate was collected by centrifugation, washed with ethyl ether (3X) and dried under high vacuum. The dried Folate-PEG₁₀₈-EDA (1.0 equiv) was treated with FITC (50 mg, 1.5 equiv) in DMSO and DIPEA at room temperature. Reaction progress was monitored by LCMS. After 10 h starting material was consumed to give the product. The crude reaction mixture was purified by preparative HPLC, (mobile phase A=10 mM Ammonium Acetate, pH=7; Organic phase B=Acetonitrile; Method: 0% B to 30% B in 35 minutes at 13 mL/min) and provided FITC-PEG108-Folate in 64% yield.

### Example 9

### Synthesis of FITC-DUPA

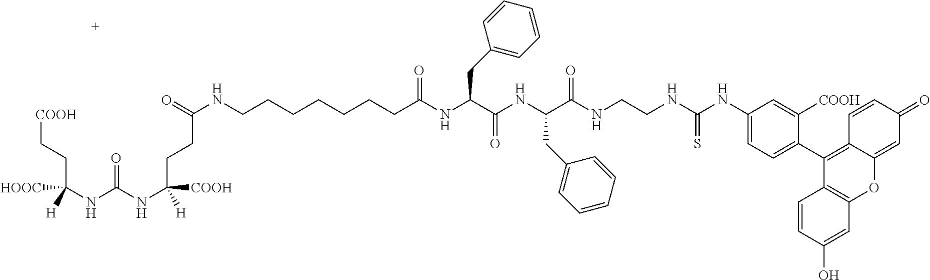 [SMILES: O=C(O)CC[C@H](NC(=O)N[C@@H](CCC(=O)NCCCCCCCC(=O)N[C@@H](Cc1ccccc1)C(=O)N[C@@H](Cc1ccccc1)C(=O)NCCNC(=S)Nc1ccc(-c2c3ccc(=O)cc-3oc3cc(O)ccc23)c(C(=O)O)c1)C(=O)O)C(=O)O]

DUPA-FITC was synthesized by solid phase methodology as follows. Universal Nova Tag™ resin (50 mg, 0.53 mM) was swollen with DCM (3 mL) followed by DMF 3 mL). A solution of 20% piperidine in DMF (3×3 mL) was added to the resin, and argon was bubbled for 5 min. The resin was washed with DMF (3×3 mL) and isopropyl alcohol (i-PrOH, 3×3 mL). After swelling the resin in DMF, a solution of DUPA-(OtBu)-OH (1.5 equiv), HATU (2.5 equiv), and i-Pr₂NEt (4.0 equiv) in DMF was added. Argon was bubbled for 2 h, and resin was washed with DMF (3×3 mL) and i-PrOH (3×3 mL). After swelling the resin in DCM, a solution of 1 M HOBt in DCM/TFE (1:1) (2×3 mL) was added. Argon was bubbled for 1 h, the solvent was removed and resin was washed with DMF (3×3 mL) and i-PrOH (3×3 mL). After swelling the resin in DMF, a solution of Fmoc-Phe-OH (2.5 equiv), HATU (2.5 equiv) and DIPEA (4.0 equiv) in DMF was added. Argon was bubbled for 2 h, and the resin was washed with DMF (3×3 mL) and i-PrOH (3×3 mL). The above sequence was repeated for 2 more coupling steps for addition of 8-aminooctanoic acid and fluorescein isothiocyanate or rhodamine B isothiocyanate. The final compound was cleaved from the resin using the Cleavage Solution and concentrated under vacuum. The concentrated product was precipitated in diethyl ether and dried under vacuum. The crude product was purified using preparative RP-HPLC [λ=488 nm; solvent gradient: 1% B to 80% B in 25 min, 80% B wash 30 min run; A=10 mM NH₄OAc, pH=7; B=acetonitrile (ACN)]. ACN was removed under vacuum, and purified fractions were freeze-dried to yield FITC-DUPA as a brownish-orange solid. RP-HPLC: tR=8.0 min (A=10 mM NH₄0Ac, pH=7.0; B=ACN, solvent gradient: 1% B to 50% B in 10 min, 80% B wash 15 min run). ¹H NMR (DMSO-d6/D₂O): S 0.98-1.27 (ms, 9H); 1.45 (b, 3H); 1.68-1.85 (ms, 11H); 2.03 (m, 8H); 2.6-3.44 (ms, 12H); 3.82 (b, 2H); 4.35 (m, 1H); 6.53 (d, J=8.1 Hz, 2H), 6.61 (dd, J=5.3, 3.5 Hz, 2H); 6.64 (s, 2H); 7.05 (d, J=8.2 Hz, 2H), 7.19 (m, 5H); 7.76 (d, J=8.0 Hz, 1H); 8.38 (s, 1H). HRMS (ESI) (m/z): (M+H)⁺ calcd for C₅₁H₅₉N₇O₁₅S, 1040.3712, found, 1040.3702. UV/vis: λ max=491 nm.

### Example 10

### Synthesis of FITC-PEG12-DUPA

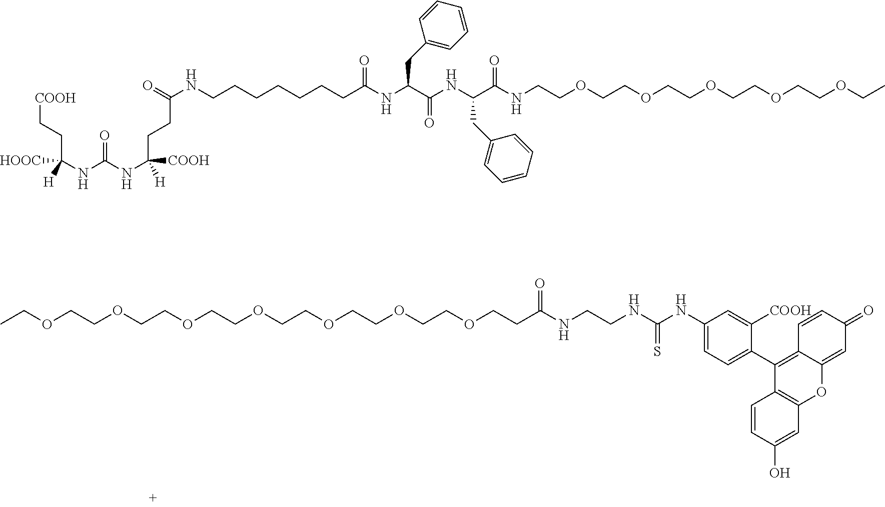

1,2-Diaminoethane trityl-resin (0.025 g) was loaded into a peptide synthesis vessel and washed with i-PrOH (3×10 mL), followed by DMF (3×10 mL). To the vessel was then introduced a solution of Fmoc-NH-(PEG)₁₂-COOH (42.8 mg) in DMF, i-Pr₂NEt (2.5 equiv), and PyBOP (2.5 equiv). The resulting solution was bubbled with Ar for 1 h, the coupling solution was drained, and the resin washed with DMF (3×10 mL) and i-PrOH (3×10 mL). Kaiser tests were performed to assess reaction progress. Fmoc deprotection was carried out using 20% piperidine in DMF (3×10 mL). This procedure was repeated to complete the all coupling steps (2×1.5 equiv of Fmoc-Phe-OH and 1.5 equiv of 8-aminooctanoic acid and 1.2 equiv of DUPA were used on each of their respective coupling steps). After the DUPA coupling, the resin was washed with DMF (3×10 mL) and i-PrOH (3×10 mL) and dried under reduced pressure. The peptide was cleaved from the resin in the peptide synthesis vessel using the Cleavage Solution. 15 mL of the Cleavage Solution was added to the peptide synthesis vessel, and the reaction was bubbled under Ar for 15 min. The resin was treated with two additional 10 mL quantities of the Cleavage Solution for 5 min each. The cleavage mixture was concentrated to about 5 mL and precipitated with ethyl ether. The precipitate was collected by centrifugation, washed with ethyl ether (3X), and dried under high vacuum, resulting in the recovery of crude material. To a stirred solution of the crude DUPA-(PEG)₁₂-EDA (10 mg) and FITC (5.6 mg) in dimethylsulfoxide (DMSO, 1 mL) was added i-Pr₂NEt (5 equiv) at room temperature and stirred for 6 h under argon. The reaction was monitored by LCMS and purified by preparative HPLC (mobile phase: A=10 mM ammonium acetate pH=7, B=ACN; method: 0% B to 50% B in 30 min at 13 mL/min). The purified fractions were pooled and freeze-dried, providing the FITC-PEG12-DUPA.

### Example 11

### Synthesis of FITC-PEG11-NK1

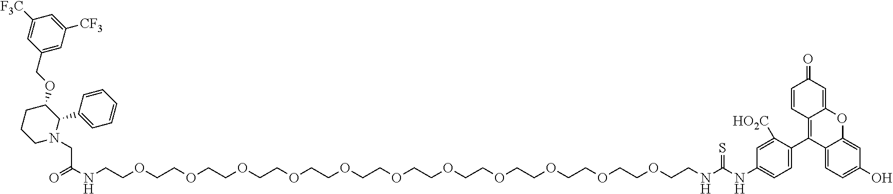 [SMILES: O=C(CN1CCC[C@H](OCc2cc(C(F)(F)F)cc(C(F)(F)F)c2)[C@@H]1c1ccccc1)NCCOCCOCCOCCOCCOCCOCCOCCOCCOCCOCCOCCNC(=S)Nc1ccc(-c2c3ccc(=O)cc-3oc3cc(O)ccc23)c(C(=O)O)c1]

To a stirred solution of NK-1 (0.02 g, 0.0433 mmol, 1.0 eq.), 0-(2-Aminoethyl)-O′-[2-(Boc-amino)ethyl]decaethylene glycol (BocNH-PEG₁₁-NH₂) (Sigma, 0.0336 g, 0.0521 mmol, 1.2 eq.), Benzotriazol-1-yl-oxytripyrrolidinophosphonium hexafluorophosphate (PyBOP) (0.027 g, 0.0521 mmol, 1.2 eq.) in dry CH₂Cl₂ was added N,N-Diisopropylethylamine (DIPEA) (0.076 mL, 0.4338 mmol, 10 eq.) under argon at room temperature. The reaction progress was monitored by LCMS and purified by preparative RP-HPLC (Waters, XBridge™ Prep C18, 5 μm; 19×100 mm column, mobile phase A=20 mM ammonium acetate buffer, pH 7, B=acetonitrile, gradient 10-100% B in 30 min, 13 mL/min, λ=220 nm, 254 nm). The pure fractions were collected, all organic solvents were evaporated and the sample was lyophilized for 48 h to provide the NK1-PEG₁₁-NHBoc. Yield: 40.13 mg (97%). To the NK1-PEG₁₁-NHBoc (0.0165 g, 0.015 mmol) in dry DCM was added trifluoroacetic acid (TFA, 20 eq.) and the reaction mixture was stirred for 4 h at r.t. The excess TFA was removed, and the remaining solution was diluted with water and extracted using CH₂Cl₂ (3×5 mL). The combined organic layers were washed with brine, dried (Na₂SO₄) and concentrated. The residue obtained was dried under vacuum and used for the next-step without further purification. A stirred solution of NK1-PEG₁₁-NH₂(0.008 g, 0.0081 mmol, 1.0 eq.), Fluorescein isothiocyanate (FITC) (Sigma, 0.0037 g, 0.0097 mmol, 1.2 eq.) in dry dimethylsulfoxide (DMSO, 0.3 mL) was added to diisopropylethyl amine (0.0028 mL, 0.0162 mmol, 2.0 eq.) at room temperature under argon. The reaction progress was monitored by LCMS and the product was purified by preparative RP-HPLC (Waters, XBridge™ Prep C18, 5 μm; 19×100 mm column, mobile phase A=20 mM ammonium acetate buffer, pH 7, B=acetonitrile, gradient 10-100% B in 30 min, 13 mL/min, λ=280 nm). The pure fractions were collected, all organic solvents were evaporated and the sample was lyophilized for 48 h to provide the FITC-PEG11-NK1 in a yield of 8.54 mg (77%).

*Note: The NK-1 compound was synthesized by a two-step procedure starting from the base ligand, which was prepared by using a procedure in the literature. (Ref: DESIGN AND DEVELOPMENT OF NEUROKININ-1 RECEPTOR-BINDING AGENT DELIVERY CONJUGATES, Application Number: PCT/US2015/44229; incorporated herein by reference.

### Example 12

### Synthesis of FITC-PEG2-CA9

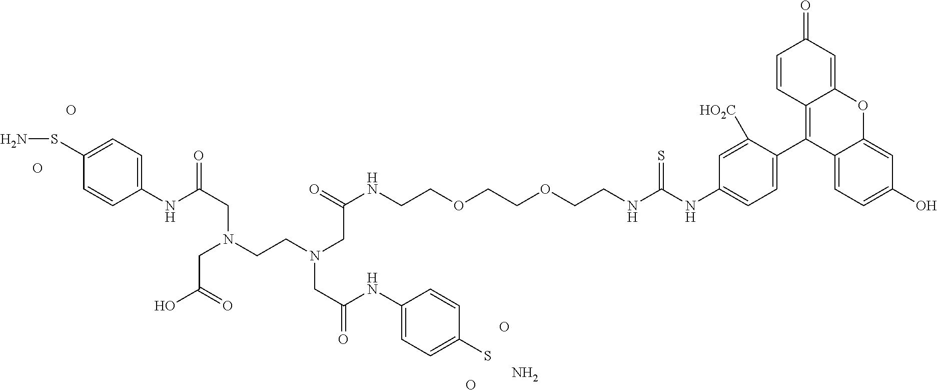

CA9 ligand (53.6 mg) was dissolved in DMF (2-3 mL) in a 50 mL round bottom flask using a Teflon magnetic stir bar. Ambient air was removed using a vacuum and replaced with nitrogen gas, this was done in three cycles. The round bottom flask was kept under constant nitrogen gas. To the flask, 28.9 mg of N-(3-Dimethylaminopropyl)-N′-ethylcarbodiimide hydrochloride (EDC) was added followed by 21.6 mg 1-Hydroxybenzotriazole hydrate (HOBt) and 18.9 μL of Boc-PEG₂-NH₂ (Sigma Aldrich). 5.4 μL of triethylamine (TEA) was added and the reaction was stirred overnight. The reaction mixture was purified using HPLC and confirmed with UHPLC-MS (target m/z of 831). Acetonitrile was removed using high vacuum rotary evaporation and the product lyopholized. The compound was mixed with 1:1 TFA:DCM for 30 minutes. The TFA/DCM was removed using high vacuum rotary evaporation followed by 30 minutes on high vacuum. The compound was then dissolved in DMF and combined with 5 molar equivalents of i-Pr₂NEt, 16 mg of fluorescein isothiocyanate (Life Technologies) and stirred for 1 h. The reaction mixture was purified by HPLC and the target compound was confirmed with UHPLC-MS (target m/z of 1120). The samples were lyophilized and stored at −20° C.

### Example 13

### Evaluation of anti-FITC scFv expression on transduced CAR-T cells

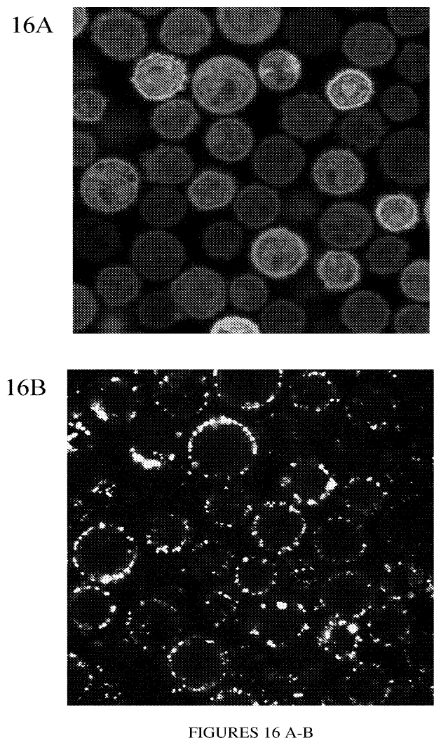

In order to confirm whether functional anti-FITC scFv is expressed on the cell surface of transduced CAR T cells, CAR T cells were incubated with FITC-Folate conjugate for 30 min on ice. After washing the cells, an anti-folate antibody (Santa Cruz) was added and incubated for 30 min on ice. Finally, Alexa Fluor 647-labeled secondary antibody (Jackson ImmunoResearch) was used to visualize CAR T cells expressing functional anti-FITC scFv. As shown in FIG. 16A, tansduced CAR T cells show GFP expression. As shown in FIG. 16B, those transduced CAR-T cells can bind FITC-Folate conjugate which was visualized by immunofluorescence labeling.

### Example 14

### Cytotoxicity assay of CAR T Cells in vitro

In order to test cytotoxicity of CAR T cells with desired FITC-ligands, a standard lactate dehydrogenase (LDH) release assay was performed using a Pierce™ LDH cytotoxicity assay kit from ThermoFisher Scientific. To prepare samples for the LDH assay, cancer cells were seeded at a density of 10⁴ cells/100 μL of media in each well of a 96 well plate and grown overnight. CAR T cells were prepared the next day at various numbers to have different effector (CAR T cell):target cell (cancer cell) ratios (e.g. E:T=20:1, 10:1, 5:1 and 1:1). FITC-ligands at various concentrations were introduced into each well and co-cultured for 6-24 hours. After co-incubation, the plate containing CAR T cells and cancer cells was centrifuged at 350×g at room temperature for 10 min to remove cell debris or remaining cells. 50 μL of the supernatants of each sample were then transferred into a new 96 well plate. 50 μL of the prepared LDH reaction mixture was added to the transferred 50 μL of each sample and incubated at room temperature for 30 min. 50 μL of stop solution was added and the absorbance of each sample was measured at 490 nm and 680 nm. The percent of cytotoxicity was calculated for each sample by using the equation below:

$$\% Cytotoxicity = ( Experimental value - effector cells spontaneous - target cells spontaneous ) / ( Target cell maximum control - target cell spontaneous control ) \times 100$$

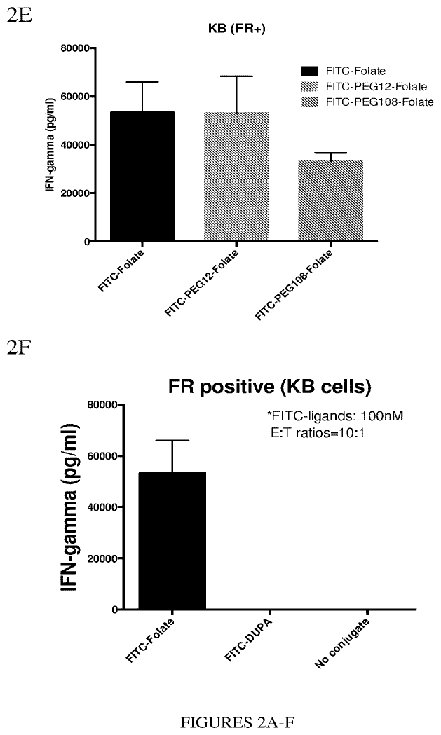

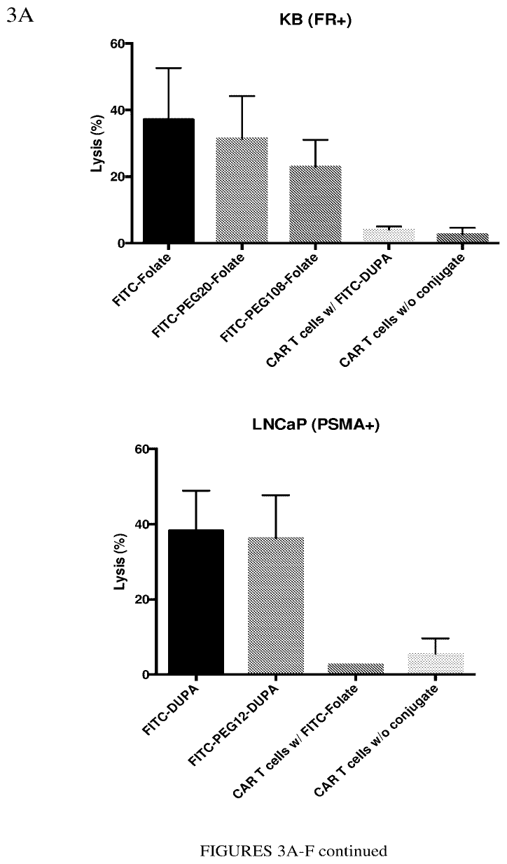

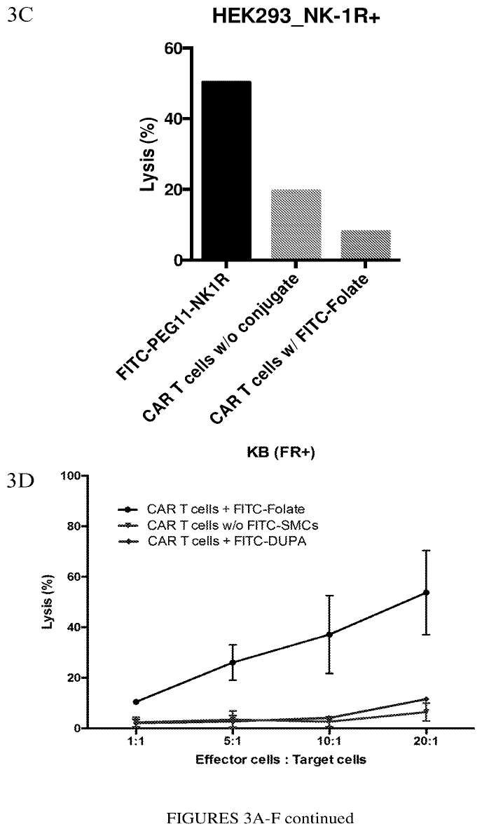

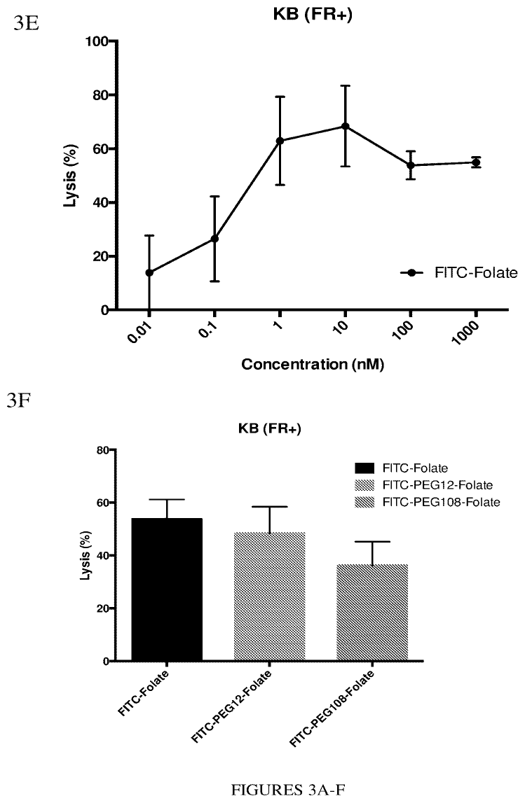

As shown in FIGS. 3A-C, CAR T cells are only activated when coupled with matching conjugates to tumor antigens. FIG. 3A shows the cytotoxicity of CART cells in a KB (FR+) model with an E:T of 10:1 using 100 nM of each conjugate. FITC-Folate, FITC-PEG20-Folate, and FITC-PEG108-Folate activation is greater than activation with FITC-DUPA or in the absence of a conjugate. FIG. 3B shows the cytotoxicity of CAR T cells in an LNCaP (PSMA+) model with an E:T of 10:1 using 100 nM FITC-DUPA or FITC-PEG12-DUPA. The FITC-DUPA conjugates show greater activation than using FITC-Folate or in the absence of a conjugate. FIG. 3C shows the cytotoxicity of CAR T cells in a HEK293 (NK1R+) model with an E:T of 10:1 using 100 nM FITC-PEG11-NK1. FITC-PEG11-NK1 shows greater than activation using FITC-Folate or in the absence of a conjugate.

As shown in FIG. 3D, CAR T cell cytotoxicity to tumor cells in a KB (FR+) model is a function of the E:T ratio used during the assay with 100 nM of FITC-Folate and FITC-DUPA. As shown in FIG. 3E, CAR T cell cytotoxicity to tumor cells in a KB (FR+) model is a function of the concentration of FITC-Folate used during the co-incubation (E:T ratio of 10:1).

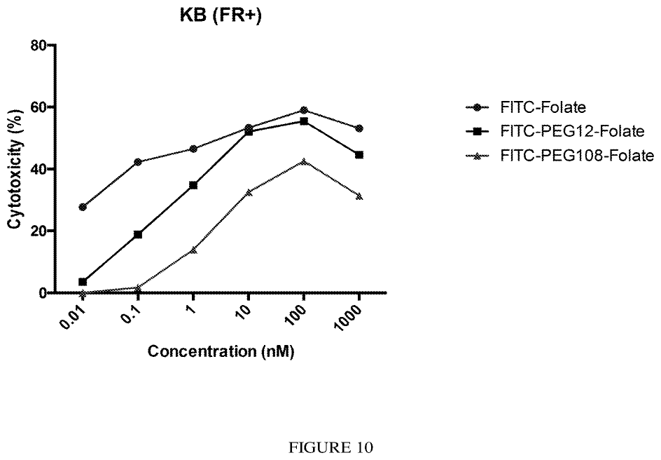

As shown in FIG. 10, CAR T cytotoxicity in a KB (FR+) model can be controlled by adjusting concentration of the conjugate bridge, or using linkers with different lengths of PEG with a E:T of 10:1.

### Example 15

### Identification of CAR T cell proliferation and CAR T cell activation

CAR T cell proliferation was mainly measured by flow cytometry. First, cancer cells (KB (FR+) or HEK (NK1R+)) were seeded at density of 10⁴ cells/100 μL of media in each well of a 96 well plate and grown overnight. The next day, CAR T cells were introduced into each well in either the presence or absence of the desired FITC-ligands and the cells were co-cultured for 5 days (120 hours). After co-incubation, CAR T cells were stained with anti-human CD3 APC antibody (Biolegend) by standard immunostaining procedures (20 min on ice). Cells positive for both anti-human CD3 staining and GFP were counted for CAR T cell proliferation.

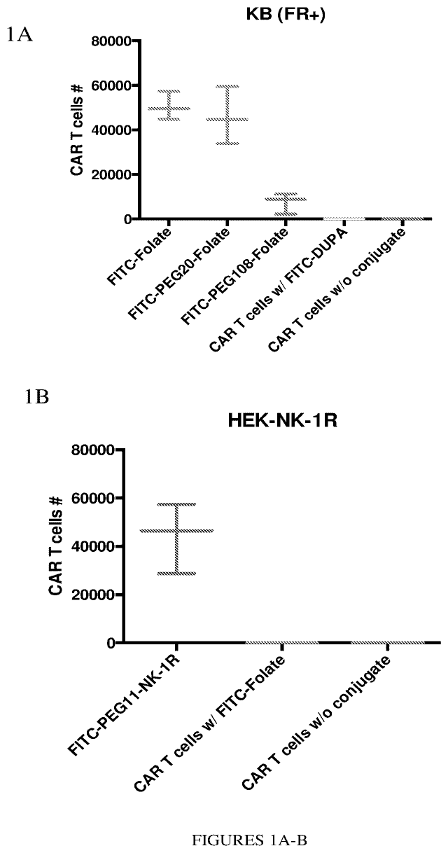

As shown in FIG. 1A-B, CAR T cells were proliferated as a result of targeting the tumor cells (E:T=5:1) with FITC-small molecular conjugate (100 nM) using KB (FR+) cells and HEK293 (NK1R+) cells. FIG. 1A shows CAR T cell proliferation in the presence of KB (FR+) cells with different conjugates. FIG. 1B shows CAR T cell proliferation in the presence of HEK (NK1R+) cells with different conjugates. As can be further seen in FIG. 1, the presence of linkers with different lengths of PEG affect the levels of CAR-T cell proliferation.

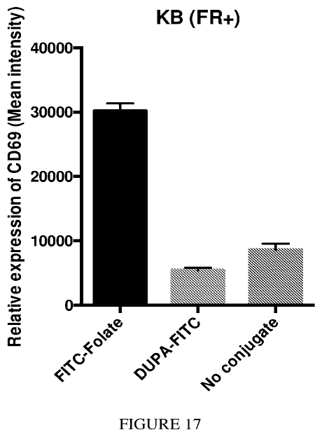

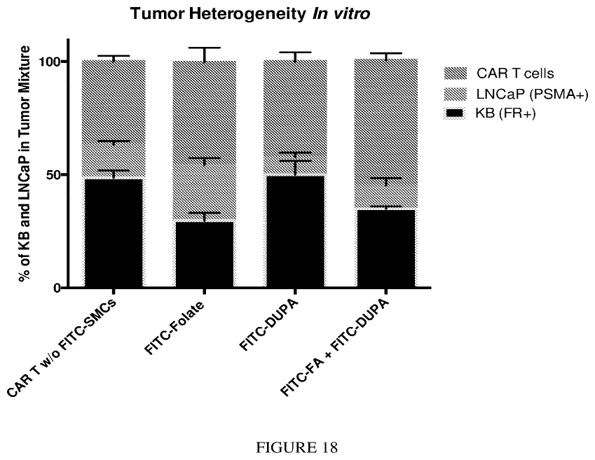

To measure CAR T cell activation, cancer cells were prepared by the same procedure described above. Cancer cells and CAR T cells were co-cultured in the presence or absence of 100 nM FITC-ligands with an E:T ratio of 10:1 for 24 hours and harvested. After washing, cells from each sample were stained with anti-human CD69 Alexa Fluor 647 (Biolegend). Data was analyzed in the gate of GFP positive T cells in order to quantify CAR T cell activation. As shown in FIG. 17, CD69 expression is related to the co-cultured conjugate.

### Example 16

### IFN-γ production assay

To test production of IFN-γ by CAR T cells, a standard ELISA assay was performed using a Human IFN-γ detection ELISA kit from Biolegend. Briefly, cancer cells were seeded at density of 10′ cells/100 μL of media in each well of a 96 well plate and grown overnight. CAR T cells were introduced into each cancer sample with desired FITC-ligands and co-cultured for 24 hours. After co-incubation, supernatants of each sample were harvested and centrifuged at 1000×g and 4° C. for 10 min to remove cell debris. Clear supernatants from each sample were then either used to detect IFN-γ by ELISA or stored at −80° C. for future usage. After completing preparation of each sample, standard ELISA was performed based on the manufacturer's instructions.

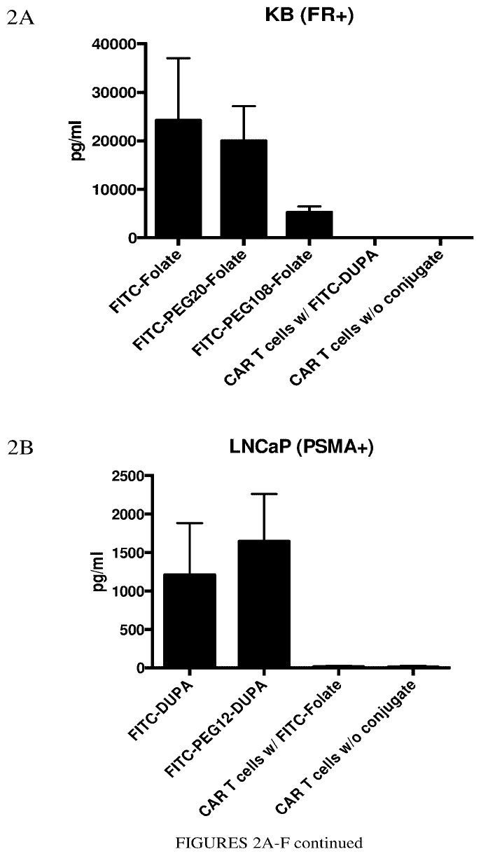

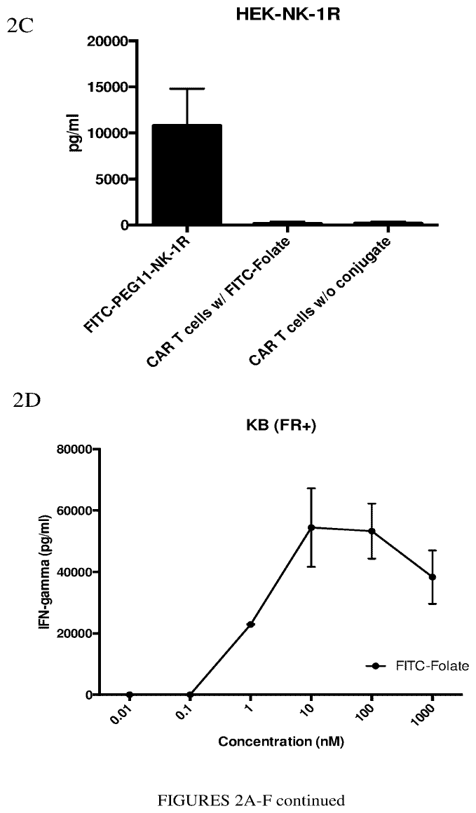

As shown in FIGS. 2A-C, with an E:T ratio of 5:1 in KB cells(FR+) (FIG. 2A), LNCaP (PSMA+) (FIG. 2B), or HEK (NK1R+) (FIG. 2C) CAR T cells produce a significant amount of inflammatory cytokine in the presence of 100 nM FITC-small molecular conjugate. As can be further seen, the amount of cytokine is dependent on the conjugate used. As shown in FIG. 2D, the amount of inflammatory cytokine is related to the concentration of FITC-Folate used with KB (FR+) cells. As shown in FIGS. 2E and 2F, the different conjugates (100 nM) produce different IFN-γ responses with KB (FR+) cells.

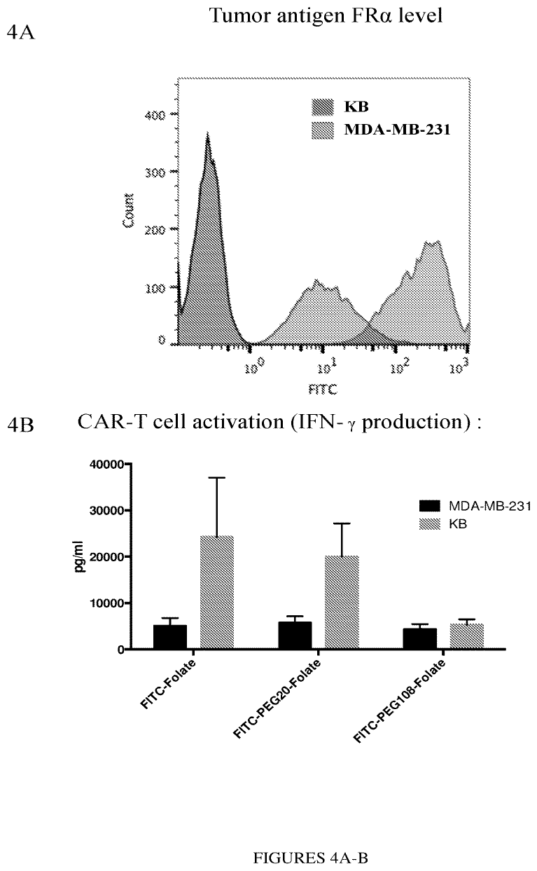

As shown in FIGS. 4A-B, activation of CAR T cells is correlated with the tumor antigen level in KB (FR+) cells and MDA-MB-231 cells. Measuring IFN-γ production from incubating the cells with same the conjugate dose shows that activation of anti-FITC CAR T cells is correlated with the expression level of tumor antigen on cancer cells.

### Example 17

### Evaluation of Correlation Between Tumor Antigen Level and CAR-T Activation

KB (FR+) cells and MDA-MB-231 cells were incubated with 100 nM FITC-folate for 30 min on ice. After washing, FITC-folate binding to FRα tumor antigen on cells was measured by flow cytometry. As shown in FIG. 4A, KB (FR+) cells have a higher level of FR expression and corresponding FITC binding than MDA-MB-231 cells.

These two cell lines were co-cultured with CAR T cells in the presence of 100 nM FITC-Folate, FITC-PEG20-Folate and FITC-PEG108-Folate with an E:T ratio of 10:1 for 24 hours. CAR T cell activation was detected by measuring its INF-y production. The supernatant of the cultured cells was harvested and the IFN-γ production was measured using an ELISA kit. As shown in FIG. 4B, with higher FRα level, KB cells (FR+) cells activated CAR T cells much better than MDA-MB-231 cells.

### Example 18

### Anti-Tumor Efficacy of CAR T Cells In Vivo

Immunodeficient NSG mice (Jackson Laboratory) were used to identify the efficacy of CAR T cell anti-tumor activity in vivo. Each tumor-specific antigen expressing cancer cell line was subcutaneously injected into the shoulder of NSG mice to establish solid tumor xenografts. When tumor volume of around 50-100 mm³ was reached, CAR T cells were introduced into mice bearing tumors and desired FITC-ligands were also introduced (i.v.) every other day. Control mice were administered PBS instead of FITC-ligands. Tumor volume and cytokine levels in the blood (IL2, IL6, IL10, IFNγ, and TNFα) were measured. General toxicity of the therapy was monitored by measuring weight loss. At the end of each treatment, mouse blood was collected to test for anemia, number of white blood cells and CAR T cell proliferation. Mouse organs were also evaluated.

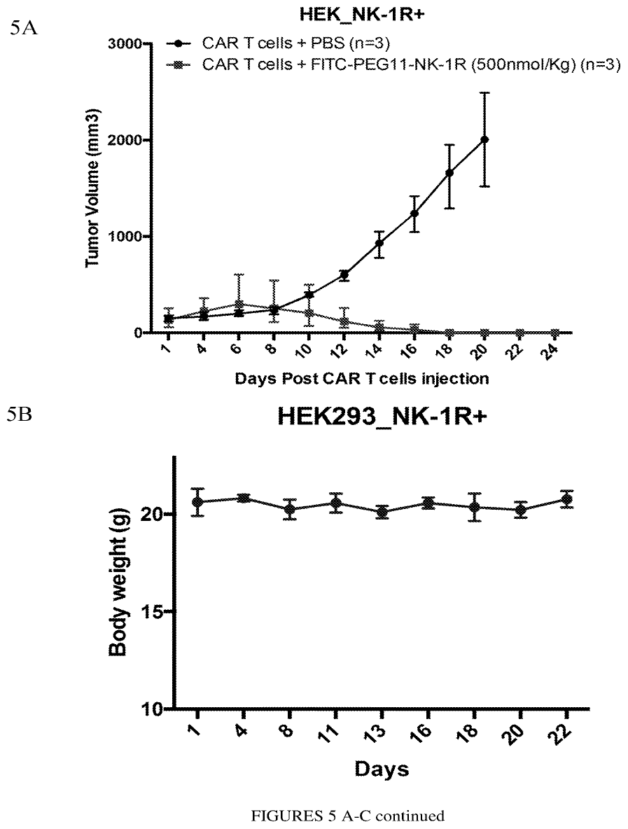

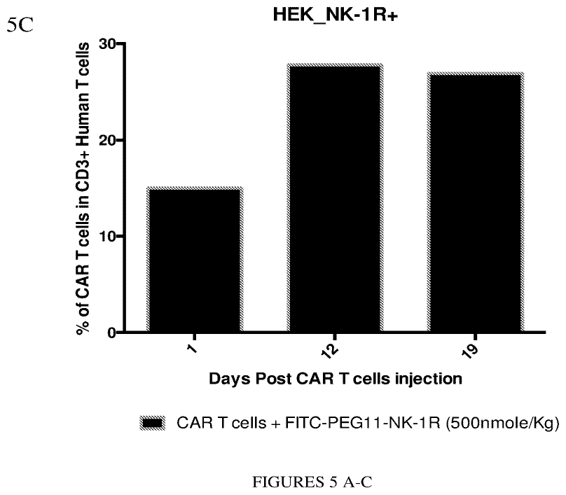

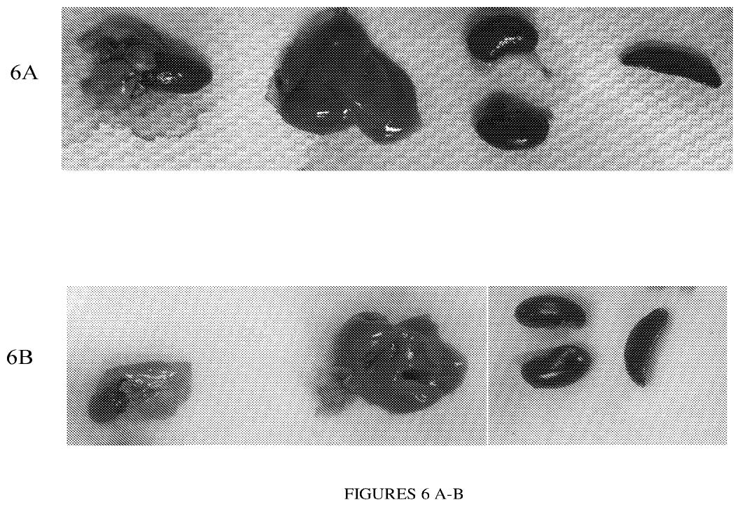

A xenograft model using HEK293 (NK1R+) cells is shown in FIGS. 5A-C and 6A-B. As shown in FIG. 5A, tumor size decreased in the mice treated with FITC-PEG11-NK1 (500 nmol/kg) but continued to grow in the control mouse. As shown in FIG. 5B, the body weight of mice treated with FITC-PEG11-NK1 was unchanged relative to the control mouse. As shown in FIG. 5C, the percentage of CART cells in human T cells increased after CART cell injection. As shown in FIGS. 6A-B, harvested organs from the treatment group (6B) appeared normal in size with no indications of cytokine release syndrome after two weeks of therapy when compared to the harvested mouse organs from a control group (6A).

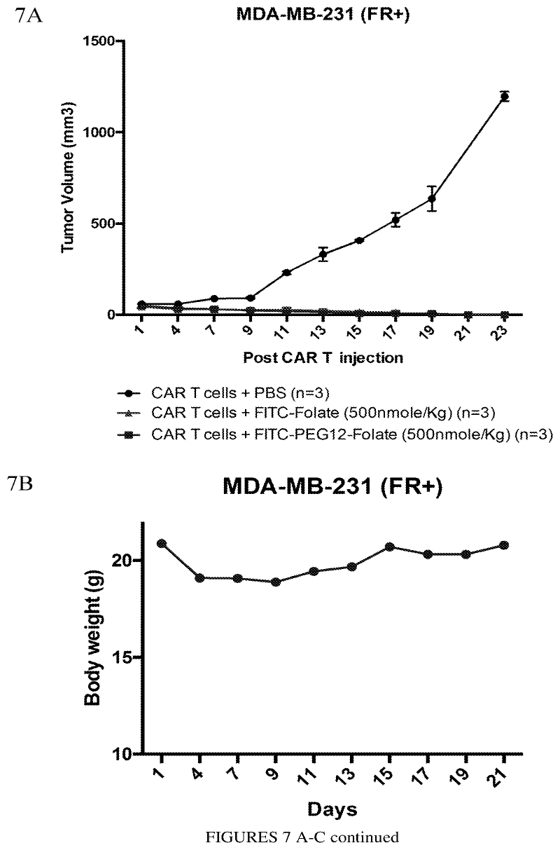

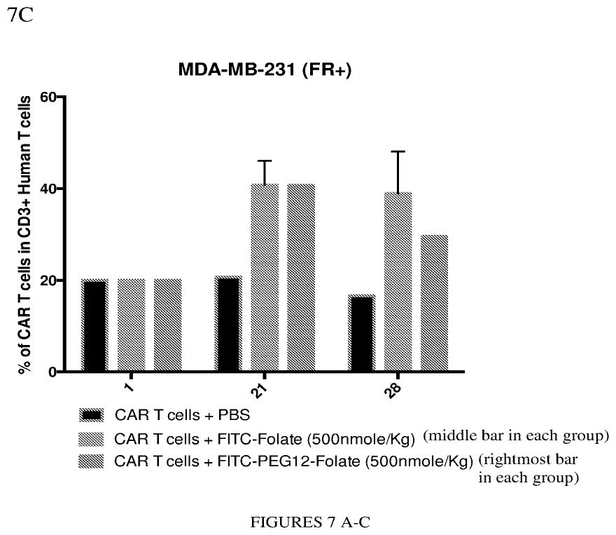

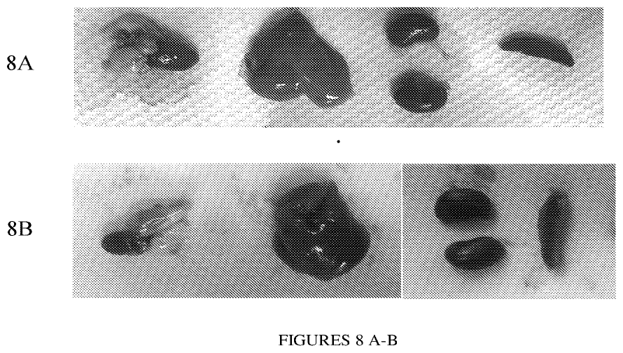

A xenograft model using MDA-MB-231 (FR+) cells is shown in FIGS. 7A-C and 8A-B. As shown in FIG. 7A, tumor size decreased in the mice treated with FITC-PEG12-Folate (500 nmol/kg) or FITC-Folate (500 nmoles/kg) but tumors continued to grow in the control mouse. As shown in FIG. 7B, the body weight of mice treated with FITC-PEG11-NK1 was unchanged during treatment. As shown in FIG. 7C, the percentage of CAR T cells in human T cells increased after CAR T cell and conjugate therapy. As shown in FIGS. 8A-B, harvested organs from the mice treated with FITC-PEG12-Folate (8B) appeared normal in size and no indication of cytokine release syndrome was observed after 3 weeks of therapy when compared to the harvested mouse organs from a control group (8A). As used in any of the embodiments in this patent application, “nmol/kg” and “nmoles/kg” are equivalent.

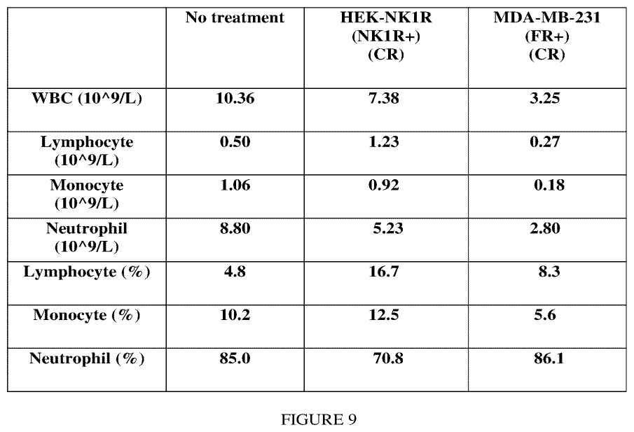

As shown in FIG. 9, blood indices of the HEK-NK1R model using FITC-PEG11-NK1 (500 nmoles/kg) and the MDA-MB-231 model using FITC-PEG12-Folate (500 nmoles/kg) also indicated that a cytokine storm had not occurred during the course of the treatment.

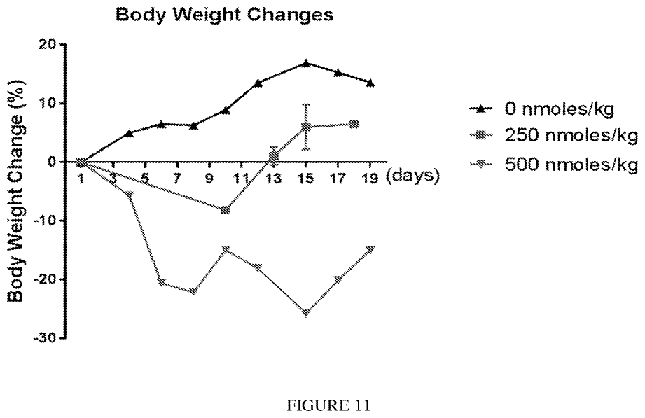

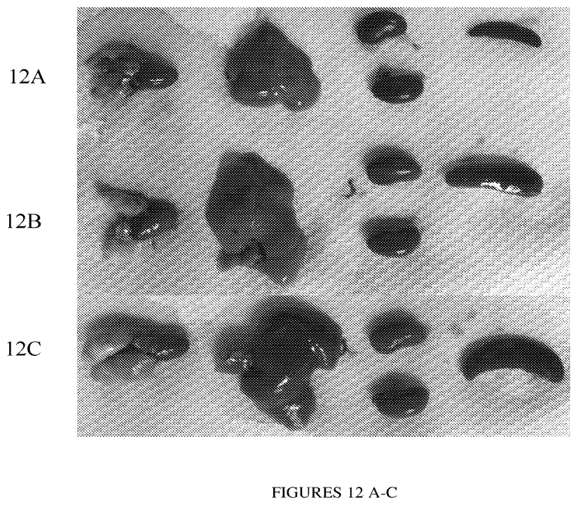

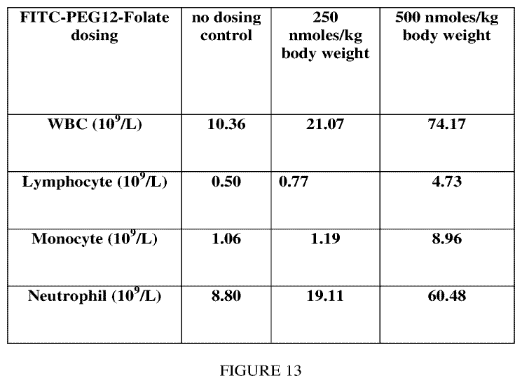

As shown in FIGS. 11, 12A-C, and 13, KB (FR+) tumor xenografts were treated with two difference concentrations of FITC-PEG12-Folate. As shown in FIG. 11, the mice treated with the lower dose (250 nmoles/kg) had milder body weight loss compared to the mice treated with a higher dose (500 nmoles/kg). As shown in FIGS. 12A-C, harvested organs from untreated mice (FIG. 12A) and mice receiving the lower dose (FIG. 12B) showed milder cytokine release syndrome compared to the higher dose (FIG. 12C). As shown in FIG. 13, KB xenograft mice receiving lower dosing showed better blood indices indicating milder cytokine release.

### Example 19

### Anti-Tumor Efficacy of CAR T Cells In Vivo

In order to study whether same anti-FITC CAR T cell can eradicate mixture of heterogeneous cancer cells with cocktail of FITC-ligands, immunodeficient NSG mice (Jackson laboratory) were utilized for the in vivo study. Two different cancer cell lines (e.g. MDA-MB-231(FR+) and HEK(NK1R+)) were implanted into separate flanks on the same mouse. Then, anti-FITC CAR T cell (10⁷ cells) was introduced by intravenous injection when tumor volumes reached about 50-100 mm³. In addition, either a mixture of FITC-ligands (i.e. FITC-PEG12-Folate (500 nmole/kg) and FITC-PEGT11-NK1R (500 nmole/kg)), single FITC-PEG11-NK-1R (500 nmole/kg) or PBS was introduced every other day by intravenous injection. The anti-tumor efficacy of CAR T cell with FITC-ligands was analyzed by measuring tumor volume at every other day.

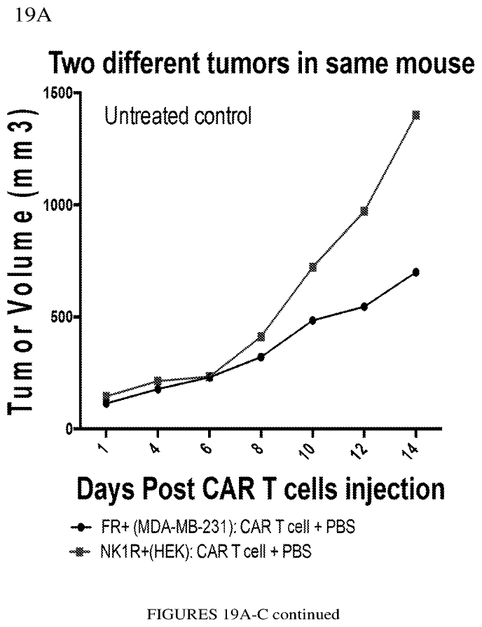

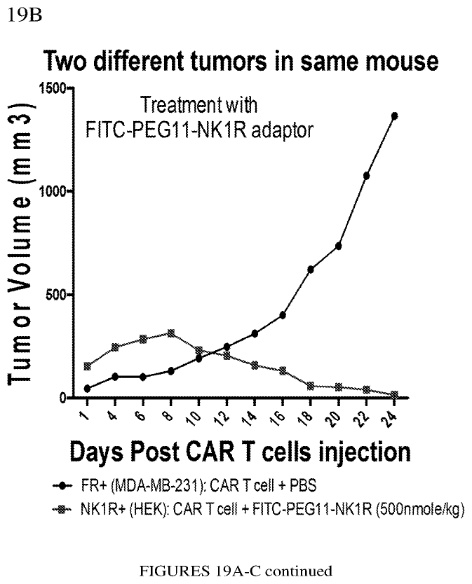

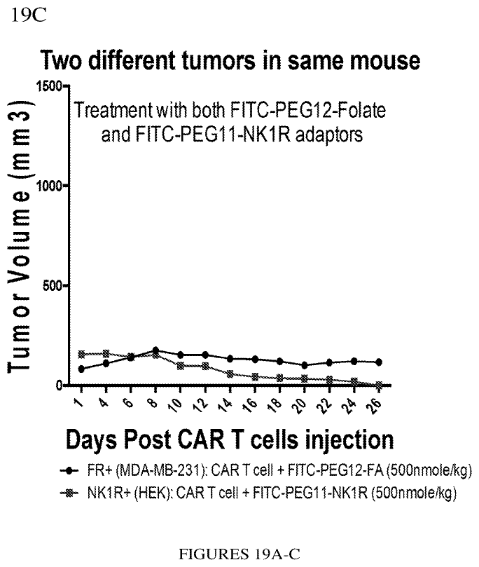

As shown in the FIGS. 19A-C, both tumors (i.e. MDA-MB-231 (MDA) and HEK (NK1R)) were eliminated by the same anti-FITC CAR T cell when both FITC-PEGT1-NK1R and FITC-PEG12-Folate were introduced. Only HEK (NK1R) tumor was eradicated in the mouse that was treated by only FITC-PEG11-NK1R. Without being bound by theory, it is believed that the MDA-MB-231 continued growing in the same mouse where only FITC-PEG11-NK1R was administered because FITC-PEG12-Folate was not administrated. As expected, both tumors did not show any response when PBS was introduced. These data suggest that the same anti-FITC CAR T cell can eradicate antigenically heterogamous tumor mixture though cocktail of FITC-ligands.

What is claimed is:

1. A method of treatment of a cancer, the method comprising,
administering to a patient a CAR T cell composition comprising a CAR T cell, wherein the CAR T cell comprises a CAR directed to a targeting moiety;
administering to the patient a first dose of a compound, or a pharmaceutically acceptable salt thereof, wherein the compound comprises a small molecule ligand linked to the targeting moiety by a linker; and
administering to the patient a second dose of the compound, or the pharmaceutically acceptable salt thereof, wherein the second dose is different than the first dose.

2. A method of treatment of a cancer, the method comprising,
administering to a patient a CAR T cell composition comprising a CAR T cell, wherein the CAR T cell comprises a CAR directed to a targeting moiety;
administering to the patient a first conjugate, or a pharmaceutically acceptable salt thereof; and
administering to the patient a second conjugate, or a pharmaceutically acceptable salt thereof,
wherein the first and the second conjugate each comprise a small molecule ligand linked to the targeting moiety by a linker and wherein the first conjugate and the second conjugate are different, wherein the small molecule ligand of the first conjugate and the second conjugate is selected from a folate, DUPA, a CCK2R ligand, and a ligand of gamma glutamyl transpeptidase.

3. A method of treatment of a cancer, the method comprising,
administering to a patient a CAR T cell composition comprising a CAR T cell, wherein the CAR T cell comprises a CAR directed to a targeting moiety;
administering to the patient a first dose of a first conjugate, or a pharmaceutically acceptable salt thereof, and
administering to the patient a second dose of a second conjugate, or a pharmaceutically acceptable salt thereof,
wherein the first conjugate and the second conjugate each comprise a small molecule ligand linked to the targeting moiety, wherein the first conjugate and the second conjugate are different, and wherein the first dose and the second dose are different, wherein the small molecule ligand of the first conjugate and the second conjugate is selected from a folate, DUPA, a CCK2R ligand, and a ligand of gamma glutamyl transpeptidase.

4. A CAR T cell comprising a nucleic acid having at least about 90 percent identity to SEQ ID NO: 1.

5. A CAR T cell comprising a polypeptide having at least about 90 percent identity to SEQ ID NO:2.

6. A nucleic acid having at least about 90 percent identity to SEQ ID NO: 1 and encoding a chimeric antigen receptor.

7. A chimeric antigen receptor polypeptide having at least about 90 percent identity to SEQ ID NO:2.

8. A vector comprising a nucleic acid having at least about 90 percent identity to SEQ ID NO: 1.

9. The CAR T cell of claim 4 comprising a nucleic acid having at least about 95 percent identity to SEQ ID NO: 1.

10. The CAR T cell of claim 5 comprising a polypeptide having at least about 95 percent identity to SEQ ID NO:2.

11. The nucleic acid of claim 6 having at least about 95 percent identity to SEQ ID NO: 1 and encoding a chimeric antigen receptor.

12. The chimeric antigen receptor polypeptide of claim 7 having at least about 95 percent identity to SEQ ID NO:2.

13. The vector of claim 8 comprising a nucleic acid having at least about 95 percent identity to SEQ ID NO: 1.

14. The CAR T cell of claim 4 comprising a nucleic acid having at least about 98 percent identity to SEQ ID NO: 1.

15. The CAR T cell of claim 5 comprising a polypeptide having at least about 98 percent identity to SEQ ID NO:2.

16. The nucleic acid of claim 6 having at least about 98 percent identity to SEQ ID NO: 1 and encoding a chimeric antigen receptor.

17. The chimeric antigen receptor polypeptide of claim 7 having at least about 98 percent identity to SEQ ID NO:2.

18. The vector of claim 8 comprising a nucleic acid having at least about 98 percent identity to SEQ ID NO: 1.

19. The CAR T cell of claim 4 comprising a nucleic acid set forth in SEQ ID NO: 1.

20. The CAR T cell of claim 5 comprising a polypeptide set forth in SEQ ID NO:2.

21. The nucleic acid of claim 6 set forth in SEQ ID NO:1 and encoding a chimeric antigen receptor.

22. The chimeric antigen receptor polypeptide of claim 7 set forth in SEQ ID NO:2.

23. The vector of claim 8 comprising a nucleic acid set forth in SEQ ID NO:1.

24. The vector of claim 8, wherein the vector is a lentiviral vector.

25. A method of treatment of a cancer, the method comprising administering to a patient the CAR T cell of claim 4, and administering to the patient a compound, or a pharmaceutically acceptable salt thereof, wherein the compound comprises a small molecule ligand linked to a targeting moiety by a linker.

26. A kit comprising at least two different types of conjugates wherein the conjugates comprise a small molecule ligand linked to a targeting moiety,
wherein the small molecule ligand in at least one of the conjugates is a CAIX ligand, and the small molecule ligand in the other conjugate is selected from a folate, DUPA, a CCK2R ligand, an NK-1R ligand, and a ligand of gamma glutamyl transpeptidase; or
wherein the small molecule ligand in at least one of the conjugates is an NK-1R ligand and the small molecule ligand in the other conjugate is selected from a folate, DUPA, a CCK2R ligand, and a ligand of gamma glutamyl transpeptidase; or
wherein the small molecule ligand in at least one of the conjugates is a ligand of gamma glutamyl transpeptidase, and the small molecule ligand in the other conjugate is selected from a folate, DUPA, a CCK2R ligand, and an NK-1R ligand.

27. The kit of claim 26, wherein the targeting moiety is FITC.

28. The method of claim 1, wherein the small molecule ligand in the compound is a CAIX ligand.

29. The method of claim 1, wherein the small molecule ligand in the compound is an NK-1R ligand.

30. The method of claim 1, wherein the small molecule ligand in the compound is folate.

31. The method of claim 1, wherein the targeting moiety is FITC.

32. A conjugate of the formula [chemical structure]
